(19) United States
(12) Patent Application Publication
(10) Pub. No.: US 2021/0065206 A1
(43) Pub. Date: Mar. 4, 2021

(54) Healthcare Malpractice Control

(71) Applicants: Carl Anthony Mecca, Rockville, MD (US); Howard Smith, Rockville, MD (US); Suman Shukla, Falls Church, VA (US); Umesh Shukla, Falls Church, VA (US)

(72) Inventors: Carl Anthony Mecca, Rockville, MD (US); Howard Smith, Rockville, MD (US); Suman Shukla, Falls Church, VA (US); Umesh Shukla, Falls Church, VA (US)

(21) Appl. No.: 17/008,420

(22) Filed: Aug. 31, 2020

Related U.S. Application Data
(60) Provisional application No. 62/893,644, filed on Aug. 29, 2019.

(51) Int. Cl. G06Q 30/00; G16H 10/20; G16H 70/20; G16H 50/70; G16H 15/00; G16H 40/20; G06Q 10/06

(52) U.S. Cl. CPC G06Q 30/018; G16H 10/20; G16H 70/20; G06Q 10/0635; G16H 15/00; G16H 40/20; G16H 50/70

(57) ABSTRACT

A method of analyzing a specific medical treatment provides efficient and effective statistical results to be included in a preponderance of evidence and/or affidavit of merit for any allegation of negligence. Following standards of care, the method creates a hypothetical treatment and determines an associated background risk of adverse outcome thereof. The method subdivides both the hypothetical treatment and the specific treatment into individual phases. By comparing the specific treatment with the hypothetical treatment in each phase, the method generates the relative risks for the phases of the specific treatment and groups them into a sample. Conducting a Student t-test with a predetermined level of confidence, the method tests a null hypothesis that the risk of an adverse outcome for the specific treatment is not significantly different from the hypothetical treatment, thus the specific treatment conforms to the standards of care. This method can help make medicine great again.

The current application claims a priority to the U.S. Provisional Patent application Ser. No. 62/893,644 filed on Aug. 29, 2019. The current application is filed on Aug. 31, 2020 while Aug. 29, 2020 and Aug. 30, 2020 were on a weekend.

### FIELD OF THE INVENTION

The present invention relates generally to healthcare data analysis. Specifically, the present invention relates to a method and system for analyzing risk data of an adverse outcome and/or injury for a specific medical treatment. The method conducts a Student t-test using the risk data to test whether or not the specific treatment conforms to the standards of care or practice guidelines determined by a user of the present invention. Thus, the present invention provides an efficient and effective method with reliable scientific results that can be used in any allegation of negligence of medical treatments.

### BACKGROUND OF THE INVENTION

A crisis is a disruptive event, but it is also a tipping point. Nothing is more disruptive to the healthcare system and the practice of medicine in the United States than are the oppressive costs of medical professional liability insurance. This was the prevailing opinion, especially, between 1983 and 2008, prompting the American Medical Association (AMA) to declare a “malpractice crisis.” It was not just about unaffordability of medical professional liability insurance; it was about the unavailability of coverage in some states because medical professional liability carriers chose to avoid doing business where there was greater claims history. It was also about the inaccessibility to important medical services, like obstetrics, in entire regions of the country. The crisis destabilized the healthcare system. Anything that destabilized healthcare, destabilized the nation, costing the United States $60 billion a year. Since conventional solutions only focused on the disruptive event and not on the tipping point, the crisis waxed and waned for almost 40 years but never really went away.

In 1983, after accelerated increases in premiums echoed in the “crisis,” some medical professional liability insurance companies responded with a solution of their own—changing from “occurrence” to “claims made” policies. Until then, “occurrence” policies were the standard and indemnified all claims occurring during the duration of coverage even if claims were reported after cancellation. “Claims made” policies appeared more affordable but provided less coverage, indemnifying only those claims reported during the duration of coverage until cancellation. Because a new “claims made” policy started with a clean slate and risks accumulated over time, “claims made,” premiums at first were low and progressed over five (5) years until the mature premium was reached. Consequently, before 2003, premiums stabilized. However, there was a problem on the back end.

Between 2003 and 2006, after many “claims made” premiums matured, premiums again increased and, in some cases, doubled. This time, the private sector responded to the crisis with their solutions—self-insurance, physician-hospital partnerships, captives, deductible plans, concierge medicine, performance-based practice and “I'm Sorry” laws. These were little more than desperate attempts to escape the crisis, had little to no impact on cost of claims and created other problems that further destabilized the climate of medicine.

Among those other problems was one that occurred when an insured dropped a “claims made” policy for any reason, such as joining a physician-hospital partnership. Before it was cancelled, most “claims made” indemnified $1 million per claim and $3 million for aggregates each year, but, afterwards, unless a “prior acts” insurance policy was purchased, there was no coverage for anything that occurred during those years but may be reported later. The statute of limitations is three (3) years and some claims had a statute of limitations of 21 years. Going bare was risky business and “prior acts” coverage was very expensive.

The most expensive solution was defensive medicine. Of the $60 billion a year, it represented $45 billion. These were costs for medical services that exceeded standards of care, offered no added quality of care and only served to protect doctors, hospitals and other organizations from potential allegations of negligence if they were not performed. These costs were totally avoidable because adherence to standards of care, alone, should be sufficient in any defense of negligence. Adding excessive services, which were neither necessary nor risk free, would not be helpful. When there were no departures from standards of care, these excesses, too, would not prevent an adverse outcome that resulted from a random occurrence that was completely unpreventable. As a solution, defensive medicine was, in and of itself, an acknowledgement of an institutionalized problem in the medical profession, which would, ultimately, make applicable standard of care into “anything goes” whenever there was the slightest insinuation of negligence.

The most practical solution was tort reform. Tort reforms did improve things but, only as much as capping punitive damages lowered cost of claims. The improvement was regional, depending on the state. The low-end cap was $250,000 per claim; most tort reforms had higher caps, which essentially did nothing, and many states had none at all.

What resulted from all these solutions was premiums remained lopsidedly high. There were some savings; however, medical professional liability carriers used most of them to build a redundancy in their reserves. Just enough savings were diverted to premiums so that they were essentially unchanged. Carriers cannot be faulted for this because, after all, their actuaries had a stake in surviving this crisis and were just doing their jobs.

Nevertheless, building this redundancy had positive results. An overview of Medical Liability Monitor data showed that, between 2008 and 2017, 74.2 percent of premiums increased no more than 10 percent over the entire time. Plotting “percent increase” of premiums on a graph showed a decreasing trend over 10 years, suggesting that the crisis had abated and medical professional liability premiums were finally stabilized. Using the same data but plotting “cost,” showed a different picture—a flat line.

Then, in 2018, 14.4% of premiums increased by an average 10 percent, implying that the stability was waning. More seriously, a higher proportion of claims incurred a greater severity of losses which boded poorly for premiums in the near future.

Asserting stability was misleading. Since 2008, the costs of this crisis were approximately the same. In 2008, premiums for doctors in high risk specialties ranged from a low of $50,000 per doctor per year to a high of $215,000. In 2018, this was basically the same. Whether then or now, it was hardly pocket change. In what world can premiums in that range be regarded as other than draconian? If a crisis in 2008, it was still a crisis today.

In the final analysis, when a disruptive event waxes and wanes, it was because the solution, that could end it, had yet to be found. Essentially, there is an underlying theme to this crisis. That theme is that the crisis is “the new normal” in which conventional solutions are insufficient and the tort system makes it entirely too easy to sue. Most private sector solutions destabilize healthcare more than they reduce the cost of claims. Tort reforms may reduce cost of claims, but not nation-wide and do little to repair the tort system. HR 1215, Protecting Access to Care Act, for instance, leaves the tort system untouched and does not preempt states with tort reforms having caps higher than $250,000 or none at all.

There is something sinister behind the new normal. The new normal has too much in common with the Cloward-Piven strategy. Both progressively destabilizes systems, like the healthcare system and the tort system. Cloward-Piven does this with the intent to collapse the entire economy and, ultimately, replace it with socialism. Of note, politicians, who frustrate solutions, like tort reforms, for this crisis, are, also, those who advance socialized medicine. Prospects for the future will remain uncertain unless and until a solution is found that is, itself, a paradigm shift. Paradigms are tipping points.

It is the objective of the present invention to provide a solution which will exert permanent and sustainable downward pressures on medical professional liability insurance premiums, malpractice litigation, and defensive medicine, thereby, entirely eliminating all their negative impacts on healthcare. This will make medicine great again.

I) The Cause—Upward Pressure on Malpractice Premiums

Although multiple variables combine to increase medical professional liability premiums, data from the General Accounting Office and other sources conclude that the only variable that statistically correlates with increasing premiums is the increasing total cost of claims. This relationship is the key to a durable solution. Tort reforms reduce total cost of claims, but not enough because they only manage punitive damages. Indeed, punitive damages are part of the crisis but are not the root cause.

Total costs of claims include avoidable and unavoidable costs. Unavoidable costs are inescapable. They arise from justified awards or settlements and the defenses of physicians that most expediently adjudicate both meritorious and non-meritorious claims of negligence. Meritorious claims, although unfortunate departures from standards of care, are anticipated in the course of professional conduct because to err is human. Non-meritorious claims, too, arise from a presumed departure, are litigated for proper purposes and are successfully defended. Justice is served. This is not the crisis.

On the other hand, avoidable costs arise from misinformed jury verdicts, damages that exceed justified awards or settlements, and the defenses of physicians in cases that go to trial even though settlements or dismissals would be more expedient. These begin with allegations of departures from standards of care. However, as the case proceeds and loses more credibility, the litigant continues on for ulterior motives. These frivolous claims are litigated for nothing more than the insinuation of a departure from standards of care. The same could be said for a frivolous defense that makes it appear that alleging a departure from standards of care is just an insinuation. Justice is only served when both a plaintiff and a defendant have their day in court for proper purposes not for miscarriages of justice. This is the crisis.

If the crisis is miscarriages in the tort system, what are their causes? Once conflicting opinions are proffered by experts on both sides of a case, total costs of claims begin to accrue for medical professional liability insurers. The difference in opinions is always the same: an iatrogenic outcome, caused by a departure from standards of care, versus an idiopathic outcome, caused by a random occurrence. Differences of opinions between medical experts are expected because opposing interpretations of the same data or facts do lead to different but valid conclusions. However, errors in their opinions, which are measurable distortions of data or facts, also cause differences and lead to invalid conclusions. Most sources of error, such as poor documentation in medical records, are knowable and correctable during discovery. One source of error, that is not so easily recognizable, is the medical expert, him or herself. The threat to validity can be an expert's competence, knowledge, bias or, more seriously, self-interest.

Error-prone opinions are a problem right from the start. In 1986, Brennan, et. al., examined 31,000 hospital records throughout New York State. Eleven hundred had diagnoses consistent with negligence from which lawsuits could be anticipated; however, only 22 (2%) of these were included in the actual 135 medical malpractice lawsuits filed within the statute of limitations. It is always possible that the remaining 113 malpractice lawsuits were missed in the initial sample, but it is also possible that erroneous expert opinions enabled some invalid claims to be filed and discouraged other valid claims from being filed.

Seen from this perspective, there is, indeed, a common denominator to miscarriages in the tort system. It is error in an expert's opinion that, ultimately, leads to filing invalid claims of negligence, unnecessary legal transactions, misinformed jury verdicts for both sides and avoidable costs. When, total costs of claims increase for medical professional liability carriers, they, in turn, increase premiums.

As simple as this may seem, error in a medical expert's opinion, and not a departure from standards of care, is the root cause of the crisis in question. Surely, something as elegant as our legal system has procedures that are designed to weed out error. There are rules for civil procedures, evidentiary hearings, interrogatories, depositions, status conferences, etc. Finally, there is the penalty of perjury. All these should discourage any intent to be deceptive. Whatever the motivation, being deceptive is still unethical. Being true to science, regardless of outcome, is what should motivate an expert. Perhaps, it is simply too easy for some medical experts, on either side of a case, to use science and disguise error as validity; this is not perjury and has no penalty under the law.

All experts must justify opinions with the certainty of “more likely than not.” To satisfy this preponderance of the evidence, at the very least, 50% plus the addition of the minutest quanta of certainty is necessary. Like it or not, this is the burden of proof. It is how a juror is instructed to understand his or her obligation when deliberating on the evidence. However, this burden of proof is a coin toss with a level of confidence that is actually uncertainty. A poorly motivated medical expert or attorney can easily distort data and still fulfill this threshold. An expert may be required to justify an opinion to the court using this burden of proof, but the expert also has a higher obligation to society when proffering the opinion.

Medical experts are scientist. Scientists eliminate error, not add it. A preponderance of the evidence may be convention for the law, it may be a judge's jury instructions, it may be how lawyers argue; it may be how juries decide; however, jurors, judges and lawyers are not scientists. The scientific standard for certainty is 95% confidence. In contrast, a preponderance of the evidence is 50.01%. This is only a judicial threshold. When an expert is 95% certain, he or she is always 50.01% certain. Undeniably, this expert can certify to fulfilling the judicial threshold. The 5% risk of error is unstated but should still be understood. This is, also, what is ethically required of an expert. Another expert, who certifies that he or she is 50.01% certain, is not necessarily 95% certain because 50.01% may be the upper limit of certainty. The 49.99% risk of error is likewise unstated. Yet, this expert, too, fulfills the burden of proof. How are jurors, judges and lawyers to know the difference between these two experts? There is a way.

Therefore, the present invention provides a solution, in this case, a method and system, which specifically concentrates on the error—prone opinion, can manage avoidable costs. The method of the present invention, in turn, can produce downward pressure on cost of claims and, thus, medical professional liability premiums.

### SUMMARY OF THE INVENTION

Although all specialty organizations have ethical guidelines for objective medical testimony, they do not focus on the principles or methodologies of this forensic decision-making process to demonstrate how and why a medical expert objectively arrived at an opinion. Thus, the first step of the method of the present invention uses the scientific method to define the heuristics for the statistical validation of an opinion. The purpose of the scientific method is to examine the influence of error on conclusions. Specific to medical malpractice, the method does so, not by testing the hypothesis that alleges negligence, called the alternative hypothesis, but, rather, by testing the hypothesis, which is always the proposition that there is no negligence, called the null hypothesis.

The method of the present invention analyzes a specific medical treatment and provides efficient and effective statistical results to be included in a preponderance of evidence and/or affidavit of merit for any allegation of negligence. The method first creates a hypothetical treatment corresponding the specific treatment following standards of care or practice guidelines. The hypothetical treatment is defined to be a safe and effective treatment with known background risk of an adverse outcome thereof. This background risk may be the inherent risk directly related to random occurrence of the adverse outcome.

The method then subdivides both the hypothetical treatment and the specific treatment into individual phases, including, but not limited to 10 phases: 1. a Presentation

Phase, 2. a Pre-Treatment Phase, 3. an Evaluation Phase, 4. a Diagnostic Phase, 5. a Discrimination Phase, 6. an Informed Consent Phase, 7. a Selection Phase, 8. a Technical Phase, 9. a Resolution Phase, 10. a Discharge Phase. By comparing the specific treatment with the hypothetical treatment, the method generates a relative risk for each phase of the specific treatment. Subsequently, the method groups the relative risk data for all phases of the specific treatment into a sample to be used in the ensuing statistical analysis.

The method conducts a one-sample Student t-test using the relative risk sample of the specific treatment. In this test, the method uses a predetermined level of confidence, alpha (α) including, but not limited to, 0.05, 0.5, etc. Additionally, the method tests a null hypothesis that the risk of an adverse outcome for the specific treatment is not significantly different from the hypothetical treatment, thus the specific treatment conforms to the standards of care. The alternative hypothesis is the opposite of the null hypothesis thus stating the specific treatment does not conform to the standards of care and may have caused malpractice, and/or injury/damage, etc. The Student t-test results in a p-value and makes further statistical conclusion based on the p-value and the predetermined. If the p-value is greater than or equal to α, the null hypothesis is accepted and the alternative hypothesis is rejected, concluding that the risk for the specific treatment is not significantly different from the hypothetical treatment, thus conforming to the standards of care. This method can help make medicine great again.

### BRIEF DESCRIPTION OF THE DRAWINGS

FIG. 1 is a block diagram illustrating the system overview of the present invention.

FIG. 2 is a flowchart for the overall process followed by the method of the present invention.

FIG. 3 is a flowchart for a sub-process for generating relative risk data of a specific medical treatment of the present invention.

FIG. 4 is a flowchart for an alternative embodiment of the sub-process for generating relative risk data of the specific medical treatment of the present invention.

FIG. 5 is a flowchart for another embodiment of the sub-process for generating relative risk data of the specific medical treatment of the present invention.

FIG. 6 is a flowchart for another embodiment of the sub-process for generating relative risk data of the specific medical treatment of the present invention.

FIG. 7 is a flowchart for another embodiment of the sub-process for generating relative risk data of the specific medical treatment of the present invention.

FIG. 8 is a flowchart for another embodiment of the sub-process for generating relative risk data of the specific medical treatment of the present invention.

FIG. 9 is a flowchart for another embodiment of the sub-process for generating relative risk data of the specific medical treatment of the present invention.

FIG. 10 is a flowchart for another embodiment of the sub-process for generating relative risk data of the specific medical treatment of the present invention.

FIG. 11 is the flowchart for another embodiment of the sub-process for generating relative risk data of the specific medical treatment of the present invention.

FIG. 12 is a flowchart for another embodiment of the sub-process for generating relative risk data of the specific medical treatment of the present invention.

FIG. 13 is a flowchart for another embodiment of the sub-process for generating relative risk data of the specific medical treatment of the present invention.

FIG. 14 is a flowchart for a sub-process for conducting a Student t-test for the relative risk data of the specific medical treatment of the present invention.

FIG. 15 is a flowchart for an alternative embodiment of the sub-process for conducting the Student t-test for the relative risk data of the specific medical treatment of the present invention.

FIG. 16 is a flowchart for an alternative embodiment of the sub-process for conducting the Student t-test for the relative risk data of the specific medical treatment of the present invention.

FIG. 17 is a flowchart for a sub-process for reporting the Student t-test results for the specific treatment of the present invention.

FIG. 18 is an equation regarding the present invention.

FIG. 19 is a table regarding total costs of litigated claims for Washington D.C. area.

FIG. 20 is a table regarding savings from two error prone plaintiff cases.

FIG. 21 is a table regarding savings from two error prone defense cases.

FIG. 22 is a table regarding avoidable cost data.

FIG. 23 is a table regarding total costs of claims of top 15 medical professional liability companies.

### DETAIL DESCRIPTIONS OF THE INVENTION

All illustrations of the drawings are for the purpose of describing selected versions of the present invention and are not intended to limit the scope of the present invention.

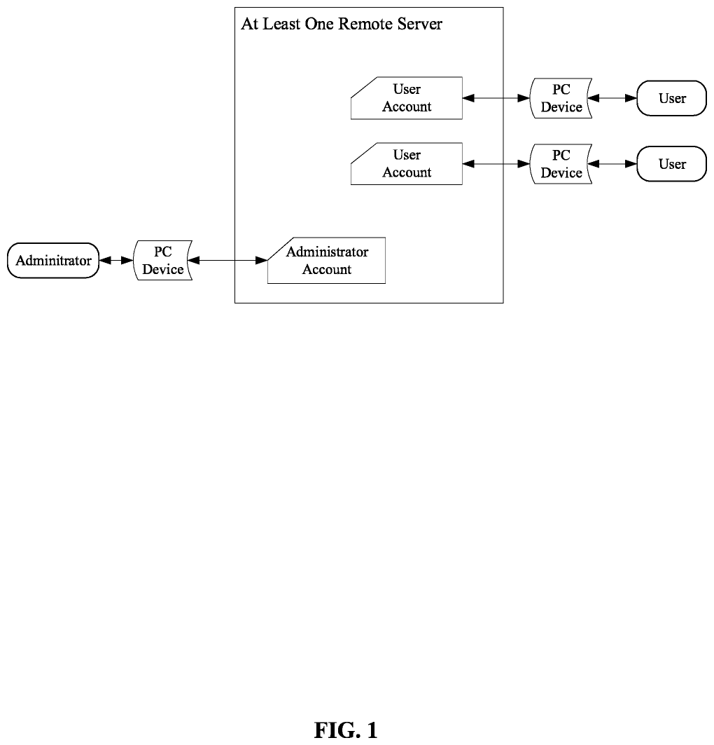

As can be seen in FIG. 1 to FIG. 17, the present invention is a system and method for providing statistical analysis of a medical treatment. The method of the present invention employs a statistical Student t-test, which fulfills and exceeds a preponderance of the evidence, to objectively prove or disprove, the null hypothesis that the occurrence of an alleged medical malpractice and/or injury equals random chance. The method thus provides scientific data that can be used in legal proceedings according to existing statutes in the law including, but not limited to, the Daubert decision that permits scientific opinions proven by the scientific method to be admissible as evidence, Rule 11 of the Federal Rules of Civil Procedure that excludes frivolous legal pleadings and penalizes those who make them, etc. The method creates a report with the statistical analysis data and affidavits of merit which certifies that expert opinions meet the burden of proof. The purpose of the method is to examine the influence of error on conclusions for a specific medical treatment. Specific to medical malpractice, the method does so, not by testing the hypothesis that alleges negligence during the treatment, called the alternative hypothesis, but, rather, by testing the hypothesis, which is always the proposition that there is no negligence, which is the null hypothesis. Thus, the present invention allows experts on both sides of a legal suite to use a standardized method to test the null hypothesis.

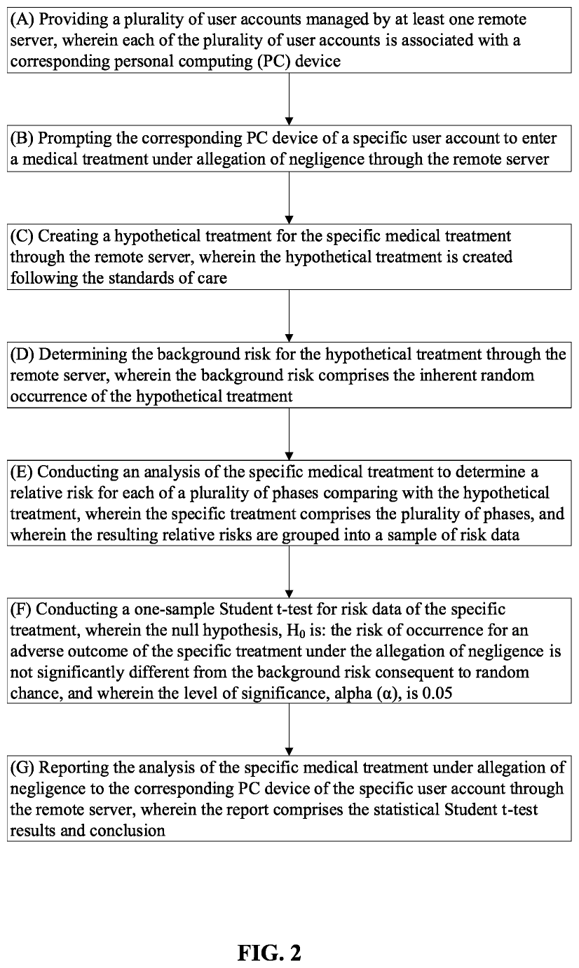

As can be seen in FIG. 1, the present invention provides an innovative system and method to report a Student t-test analysis results associated with an actual medical treatment in question or allegation of negligence to a user. To accomplish this, the method of the present invention associates each of the plurality of users with a unique user account from a plurality of user accounts that are managed by at least one remote server (Step A), as seen in FIG. 2. Each of the plurality of user accounts is associated with a corresponding personal computing (PC) device. The corresponding PC device allows a user to interact with the present invention and can be, but is not limited to, a smartphone, a smart watch, a cloud PC, a laptop, a desktop, a server, a terminal PC, or a tablet PC, etc. The users of the user accounts may include relevant parties such as, but are not limited to, individuals, medical experts, medical practitioners, healthcare professionals, physicians, doctors, nurses, healthcare administrators, medical assistants, office managers, medical providers, healthcare insurers, medical insurance providers, legal practitioners, layers, attorneys, judges, court clerks, officials, managers, business owners, consumers, companies, corporations, hospitals, medical clinics, healthcare offices, insurance companies, medical associations, healthcare associations, government entities, administrators, etc. Further, the at least one remote server is used to manage the medical treatment analysis platform for the plurality of user accounts. The remote server can be managed through an administrator account by an administrator as seen in FIG. 1. The administrator who manages the remote server includes, but is not limited to, owner, service provider, any listed user that can access the application in compliance with the user agreement and governed by that agreement, medical professional, medical expert, medical office manager, technician, engineer, system engineer, system specialist, software engineer, information technology (IT) engineer, IT professional, IT manager, IT consultant, service desk professional, service desk manager, consultant, manager, executive officer, chief operating officer, chief technology officer, chief executive officer, president, company, corporation, organization, etc. Moreover, the remote server is used to execute a number of internal software processes and store data for the present invention. The software processes may include, but are not limited to, server software programs, web-based software applications or browsers embodied as, for example, but not limited to, websites, web applications, desktop applications, cloud applications, and mobile applications compatible with a corresponding user PC device. Additionally, the software processes may store data into internal databases and communicate with external databases, which may include but are not limited to medical databases, healthcare databases, standards of care databases, databases maintaining data about medical/healthcare practices, databases maintaining regulations/laws regarding medical treatment, etc. The interaction with external databases over a communication network may include, but is not limited to, the Internet.

As can be seen in FIG. 2, the overall process of the method of the present invention prompts the corresponding PC device of a specific user account to enter a medical treatment under allegation of negligence through the remote server (Step B). More specifically, the method interacts with the PC device of the specific user account to receive a request of conducting a statistical Student t-test for the specific medical treatment. The specific medical treatment entered by the user may be, but is not limited to, an actual medical treatment in question, a medical treatment in allegation of negligence, etc. Additionally, the specific medical treatment may include, but is not limited to, malpractice, injury due to malpractice, departure from standards of care, violation of any regulations and/or laws, etc. Subsequently, the method creates a hypothetical treatment for the specific medical treatment through the remote server, wherein the hypothetical treatment is created following the standards of care or practice guidelines (Step C). More specifically, the method uses existing standards of care to create a hypothetical treatment which is a safe and effective treatment. This hypothetical treatment represents the duty that all physicians owe to perform within standards of care for the specific medical treatment in question. Further, the method determines the background risk for the hypothetical treatment through the remote server, wherein the background risk comprises the inherent random occurrence of the hypothetical treatment (Step D). Even a safe and effective treatment can result in an idiopathic outcome caused by random chance that is the same as an iatrogenic outcome caused by a departure from standards of care. The incidence of the idiopathic occurrence is the background risk in a normal population of existing numerous treatments that followed standards of care. The background risk is the statistical mean, μ, of this population of data. The background risk for most conditions can be researched in the medical literature. For example: the incidence of acute intestinal obstruction following any abdominal surgery is 0.69%.

Subsequently, the method conducts an analysis of the specific medical treatment to determine a relative risk for each of a plurality of phases comparing with the hypothetical treatment, wherein the specific treatment comprises the plurality of phases, and wherein the resulting relative risks are grouped into a sample of risk data (Step E). The method prepares an analysis of the specific medical treatment, which is the actual treatment rendered by the user. Often, there is a discrete seminal event that discriminates the specific treatment from the hypothetical treatment; other times there is not. For example, in the case of intestinal obstruction following abdominal surgery, scar tissue may adhere bowel together resulting in some modification of standard technique. Actual treatment that is different from the hypothetical treatment does not necessarily imply that there is a breach of duty to perform within the standards of care. Judgment should be reserved over these differences until the results are tested by the present invention.

Once the sample of risk data is obtained, the method conducts a one-sample Student t-test for risk data of the specific treatment, wherein the null hypothesis, H₀, is: the risk of occurrence for an adverse outcome of the specific treatment under the allegation of negligence is not significantly different from the background risk consequent to random chance, and wherein the level of significance, alpha (α), is 0.05 (Step F). The method states the null hypothesis, Hₒ, as: “Under the circumstances specific to the allegation of negligence, risk of the occurrence for an adverse outcome consequent to treatment is not significantly different from background risk of its occurrence consequent to random chance. Therefore, the adverse outcome was unpreventable, not a result of the specific treatment, and not negligence.” In terms of the scientific method, Hₒ=μ. The method tests the null hypothesis. The one sample t-test compares the mean score found in an observed sample to a hypothetically assumed value, the population mean. With the use of continuous measurements (risks), independent variables (the sample), a dependent variable (the alleged injury), and a known, the background risk, μ, the one-sample Student t-test is appropriate for hypothesis testing in the method of the present invention. The Student t-test, itself, is part of most standard statistics software. For example, relative risks of nine variables of 0.69% and one variable of 0.7245% are entered in the sample. The background risk, 0.69%, is entered for null hypothesis or μ. The value entered for the level of significance, or alpha, α, is 0.05, the scientific standard and the default level of significance for most statistical testing. This indicates that the risk of rejecting a true null hypothesis, called a type-1 error, is 5%. In contrast, a preponderance of the evidence is 50% plus some quanta or α=0.5. This corresponds to a 50% chance of making a type 1 error. The upper tail is selected because the alternative hypothesis infers the risk from treatment is greater than the background risk from chance.

Further, the method reports the analysis of the specific medical treatment under allegation of negligence to the corresponding PC device of the specific user account through the remote server, wherein the report comprises the statistical Student t-test results and conclusion (Step G). The result is called the p-value, or the observed level of significance. The p-value is the lowest level of significance at which the null hypothesis can be rejected for a given sample. If the p-value is greater than or equal to 0.05, α, the null hypothesis is retained and there is 95% confidence that there is no correlation between the independent variables and the injury or specific treatment in question. If the p-value is less than 0.05, the null hypothesis is rejected and there is a 95% confidence that the variables are statically significant to the injury or specific treatment. In the example, the p-value is 0.173297. The null hypothesis is retained. Therefore, the increase in risk for the specific treatment did not change the outcome of this as being a random occurrence. This result has a 95% level of confidence and only a 5% risk of making a type 1 error. In contrast, if a is 0.5, the null hypothesis is rejected, but the level of confidence is only 50% and there is a 50% risk of making a type 1 error. Once completed, the method prepares a report or an affidavit of merit which legally certifies that the opinion fulfills the burden of proof, is scientifically valid, has been and can be tested for validity with a specified level of significance (0.05 or 0.5 as the case may be), is consistent with peer review and accepted science and has been objectively examined for error.

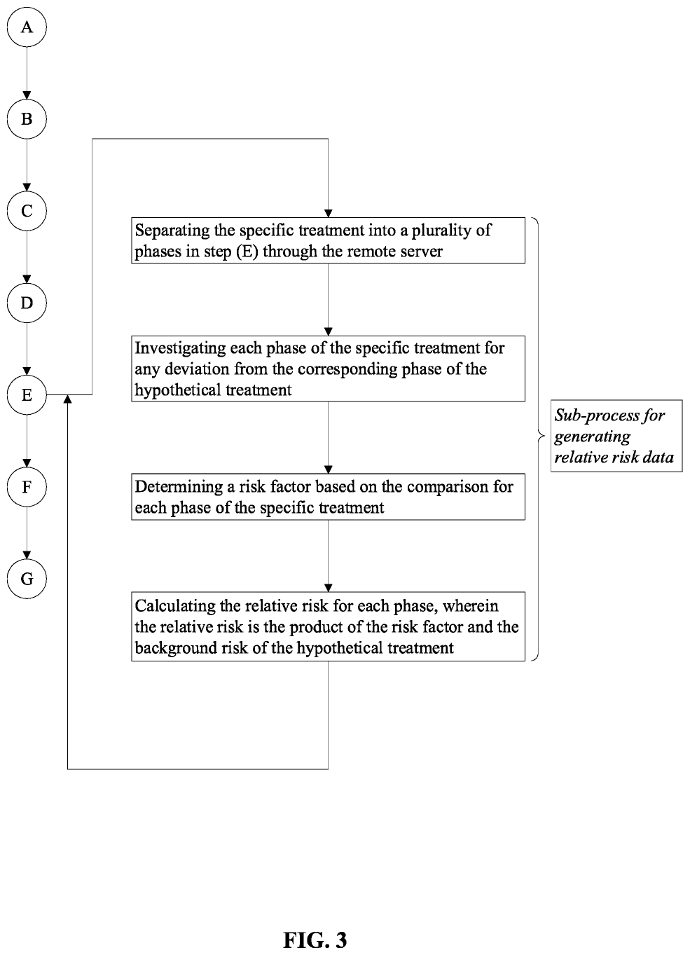

As can be seen in FIG. 3, the method of the present invention provides a sub-process for generating relative risk data used in the Student t-test for the specific treatment. More specifically, the method separates the specific treatment into a plurality of phases in Step E through the remote server. Subsequently, the method investigates each phase of the specific treatment for any deviation from the corresponding phase of the hypothetical treatment, and determines a risk factor based on the comparison for each phase of the specific treatment. Further, the method calculates the relative risk for each phase, wherein the relative risk is the product of the risk factor and the background risk of the hypothetical treatment. In the preferred embodiment of the present invention, the method separates both the hypothetical and the specific treatments into ten phases: 1. a Presentation Phase, 2. a Pre-Treatment Phase, 3. an Evaluation Phase, 4. a Diagnostic Phase, 5. a Discrimination Phase, 6. an Informed Consent Phase, 7. a Selection Phase, 8. a Technical Phase, 9. a Resolution Phase, 10. a Discharge Phase. Of note is a codicil for the Informed Consent Phase. For example, there are times when a patient has a pre-existing condition, known, such as previous surgery, or unknown, such as a congenital malformation, which is unrelated to the treatment per se, but, when discovered during treatment, can alter the Technical Phase and increase the background risk of the outcome in question. This may be the case if a history of previous abdominal surgery or some preexisting condition is uncovered prior to abdominal surgery. If such circumstances are foreseeable by any prudent practitioner, they are to be included in the Informed Consent Phase. In this example, documentation of such a discussion in the medical record satisfies the codicil.

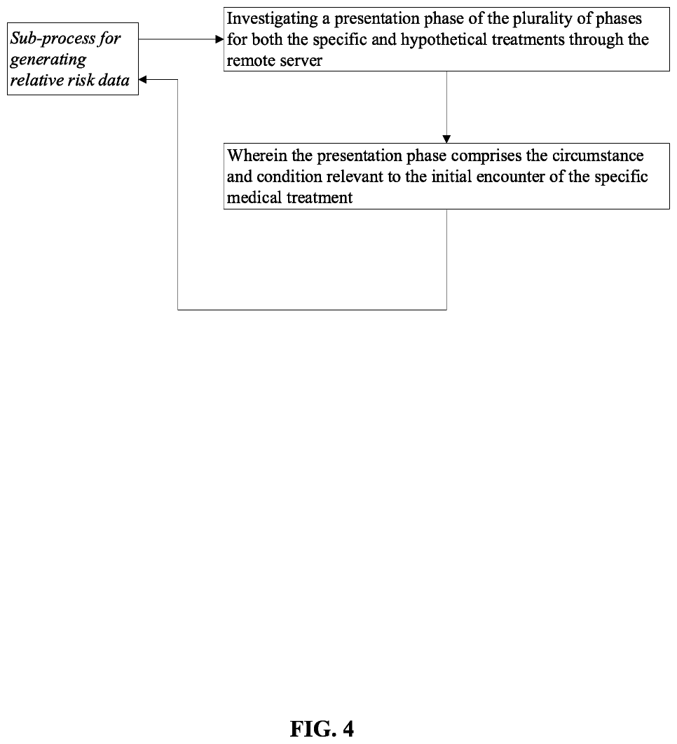

As can be seen in FIG. 4, the method investigates a presentation phase of the plurality of phases for both the specific and hypothetical treatments through the remote server, wherein the presentation phase comprises the circumstance and condition relevant to the initial encounter of the specific medical treatment.

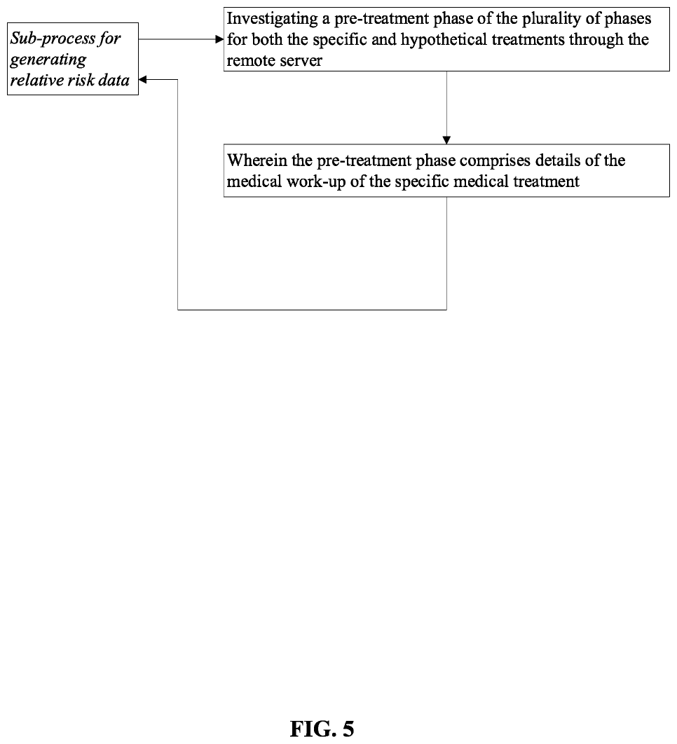

As can be seen in FIG. 5, the method investigates a pre-treatment phase of the plurality of phases for both the specific and hypothetical treatments through the remote server, wherein the pre-treatment phase comprises details of the medical work-up of the specific medical treatment.

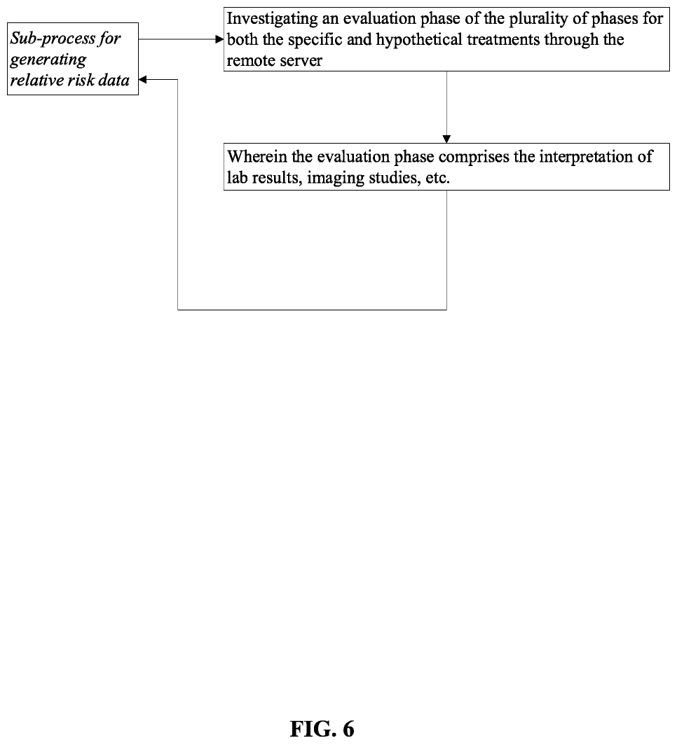

As can be seen in FIG. 6, the method investigates an evaluation phase of the plurality of phases for both the specific and hypothetical treatments through the remote server, wherein the evaluation phase comprises the interpretation of lab results, imaging studies, etc.

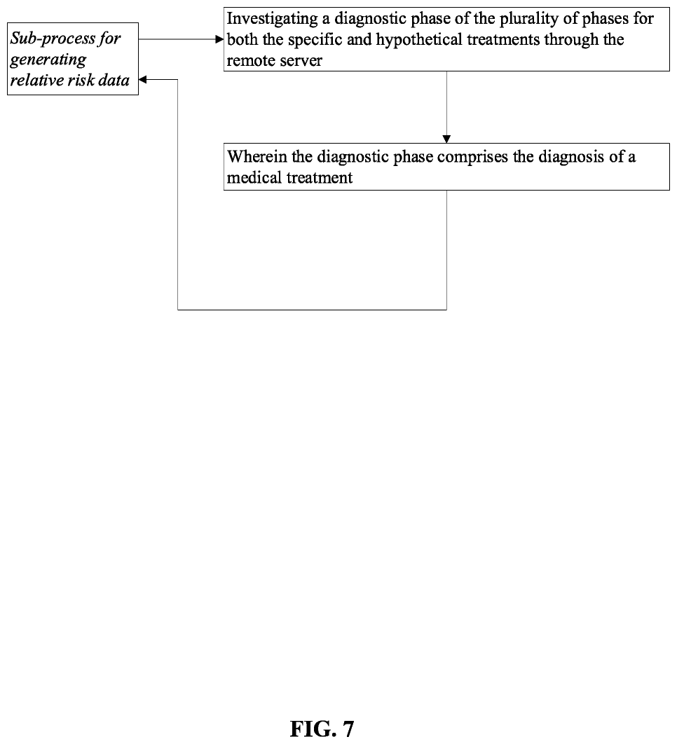

As can be seen in FIG. 7, the method investigates a diagnostic phase of the plurality of phases for both the specific and hypothetical treatments through the remote server, wherein the diagnostic phase comprises the diagnosis of a medical treatment.

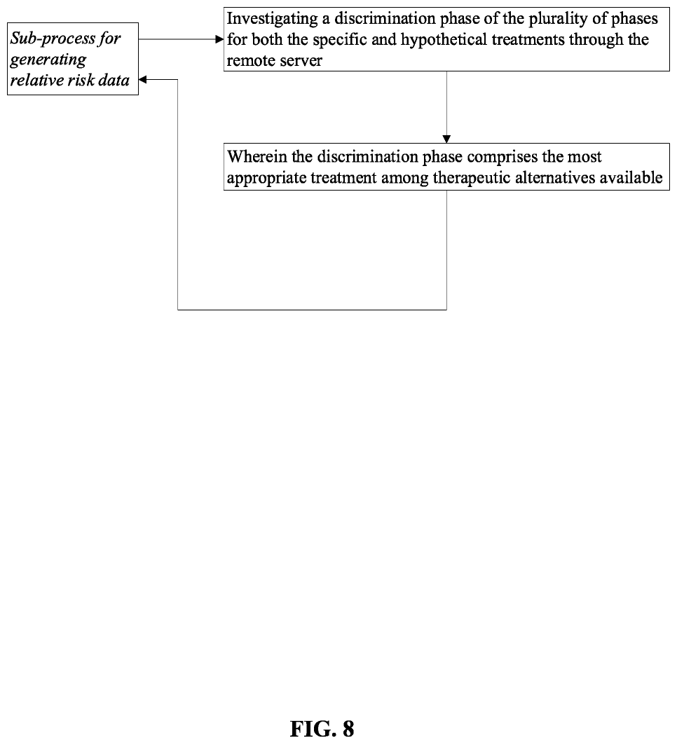

As can be seen in FIG. 8, the method investigates a discrimination phase of the plurality of phases for both the specific and hypothetical treatments through the remote server, wherein the discrimination phase comprises the most appropriate treatment among therapeutic alternatives available.

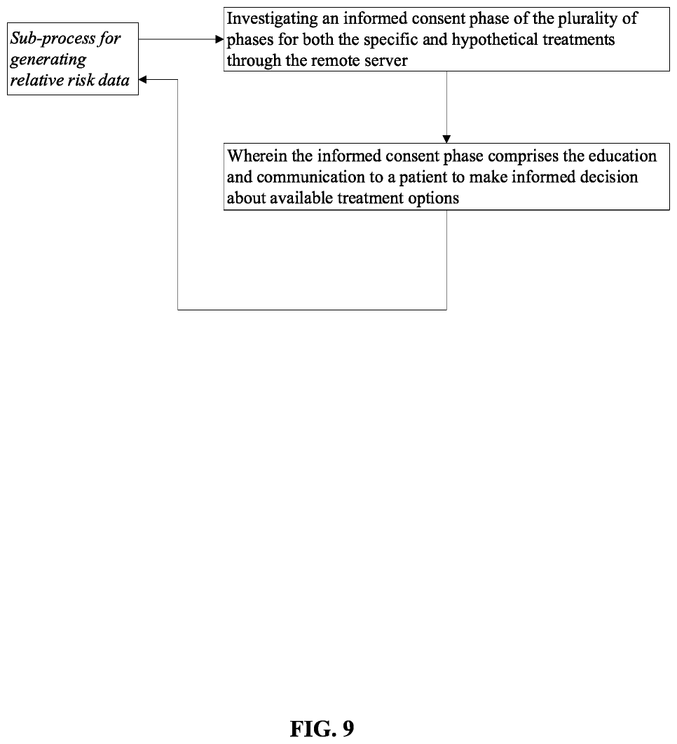

As can be seen in FIG. 9, the method investigates an informed consent phase of the plurality of phases for both the specific and hypothetical treatments through the remote server, wherein the informed consent phase comprises the education and communication to a patient to make informed decision about available treatment options.

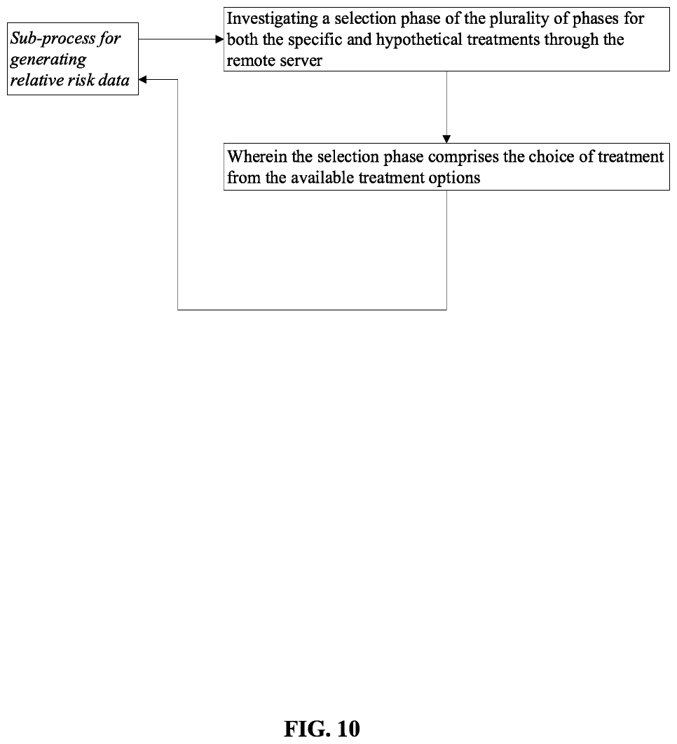

As can be seen in FIG. 10, the method investigates a selection phase of the plurality of phases for both the specific and hypothetical treatments through the remote server, wherein the selection phase comprises the choice of treatment from the available treatment options.

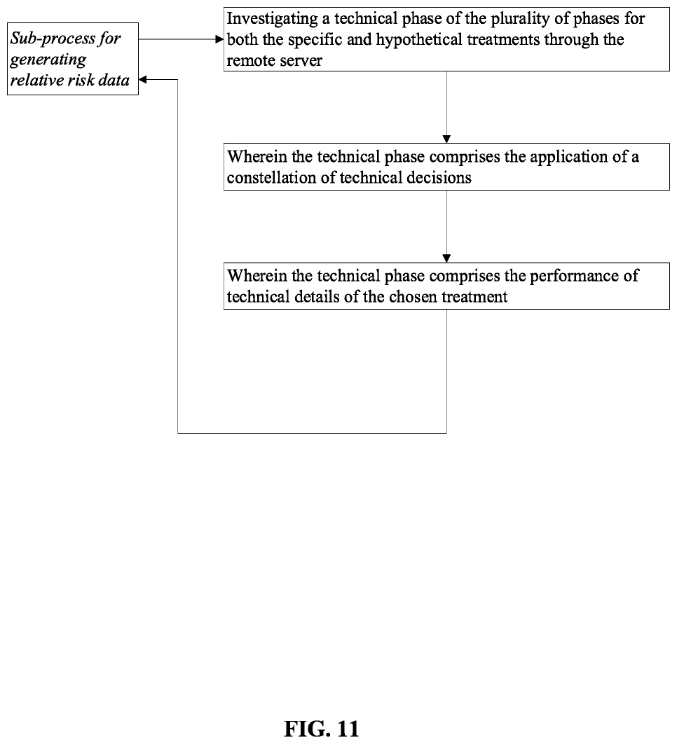

As can be seen in FIG. 11, the method investigates a technical phase of the plurality of phases for both the specific and hypothetical treatments through the remote server, wherein the technical phase comprises the application of a constellation of technical decisions, and wherein the technical phase comprises the performance of technical details of the chosen treatment.

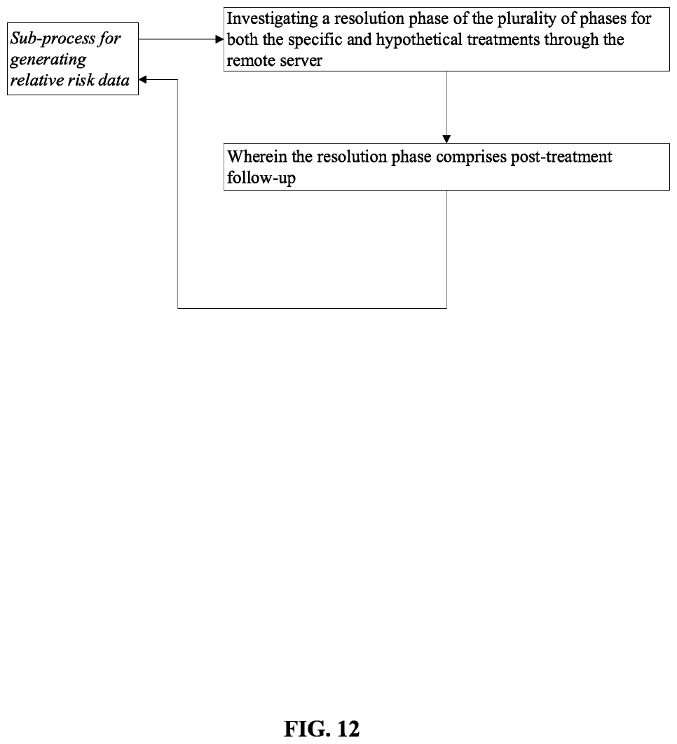

As can be seen in FIG. 12, the method investigates a technical phase of the plurality of phases for both the specific and hypothetical treatments through the remote server, wherein the resolution phase comprises post-treatment follow-up.

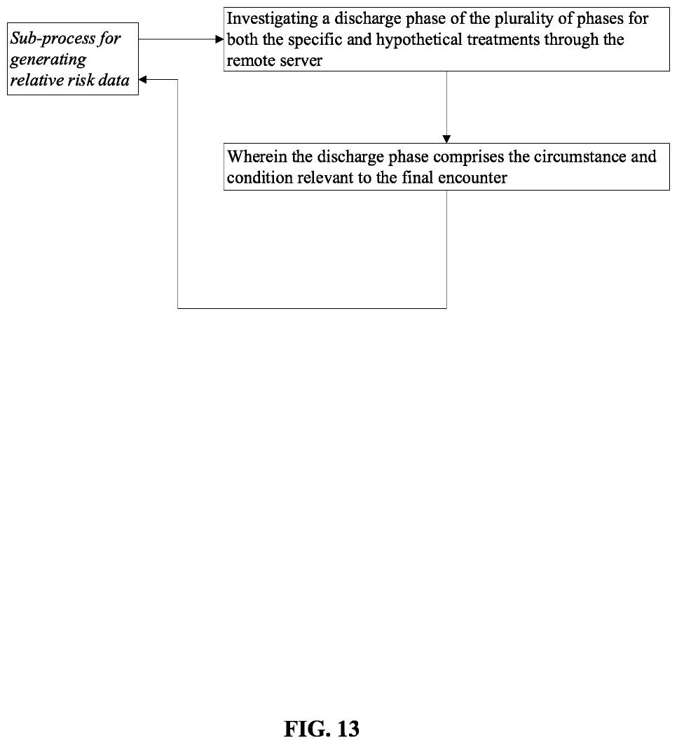

As can be seen in FIG. 13, the method investigates a discharge phase of the plurality of phases for both the specific and hypothetical treatments through the remote server, wherein the discharge phase comprises the circumstance and condition relevant to the final encounter.

The method determines the relative risk for each of the 10 phases in the hypothetical treatment never exceeds 100% of the background risk because it is the safest effective treatment. It has a relative risk of 1.0. Each phase in the specific treatment is compared to its counterpart in the hypothetical treatment to determine if the risk imposed by that phase is the same or higher with the rationale for increasing the risk and being documented by the method in the analysis. In the example chosen, the method determined that the risk in the Technical Phase, is 5% higher than the counterpart in the hypothetical treatment having a relative risk of 1.05. The method documents that the risk is 5% higher because, although this surgeon encountered these findings in other surgeries and was aware of finding them in this case, lapses of technique are always possible. The product, 1.05×0.69%=0.7245%, is the actual probability, or observed relative risk, for the occurrence of the adverse outcome. When these calculations are completed for the 10 phases of the specific treatment, the results, essentially, are a sample of ten observed risks, nine of 0.69% and one of 0.7245%, relating the medical treatment of this patient to the occurrence of the adverse outcome. These are all independent variables. Although one or more observed risks might appear higher than the background risk, again, judgments should be reserved until the results are tested.

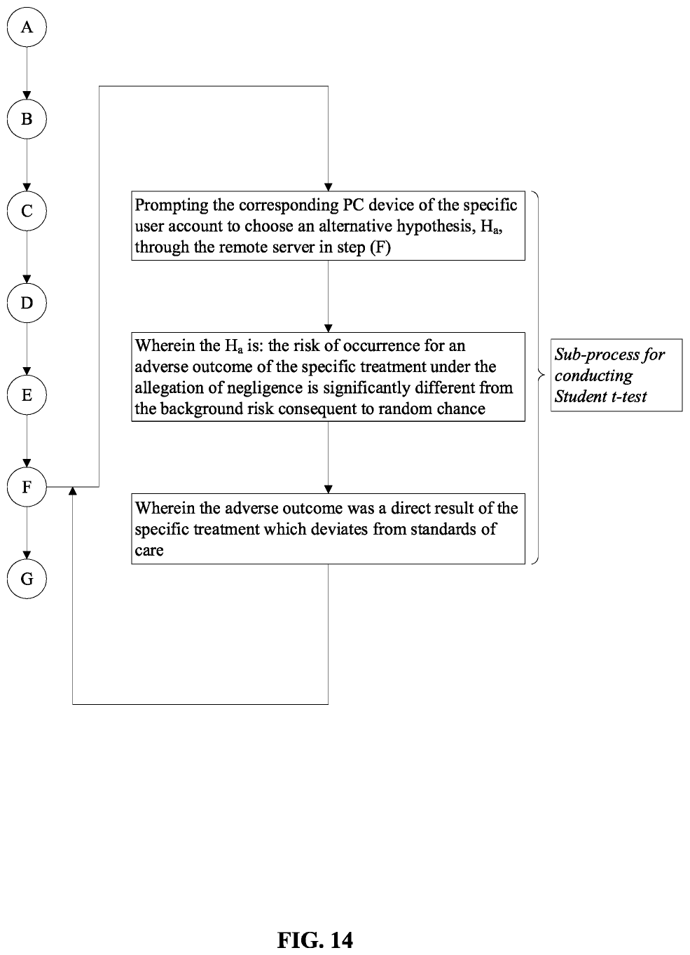

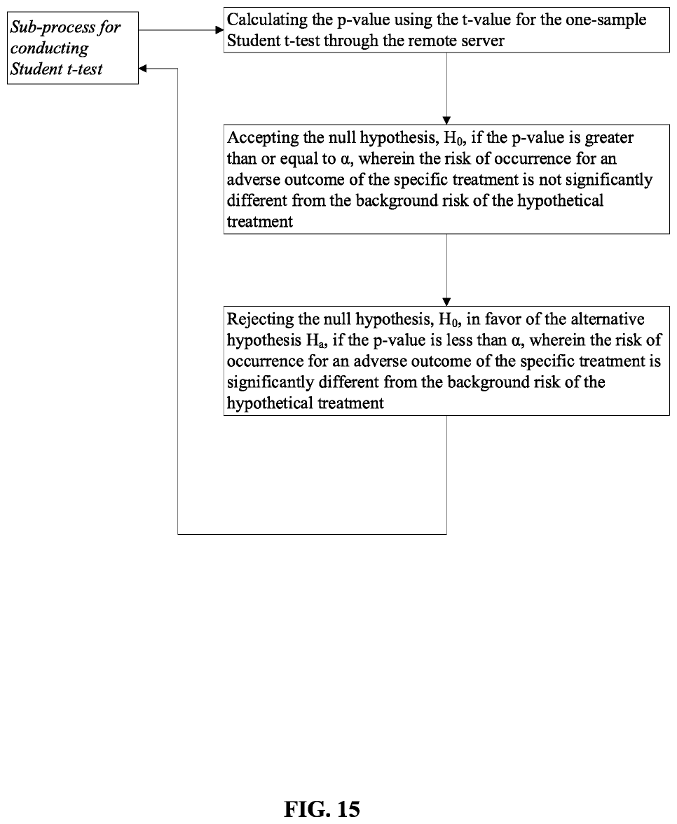

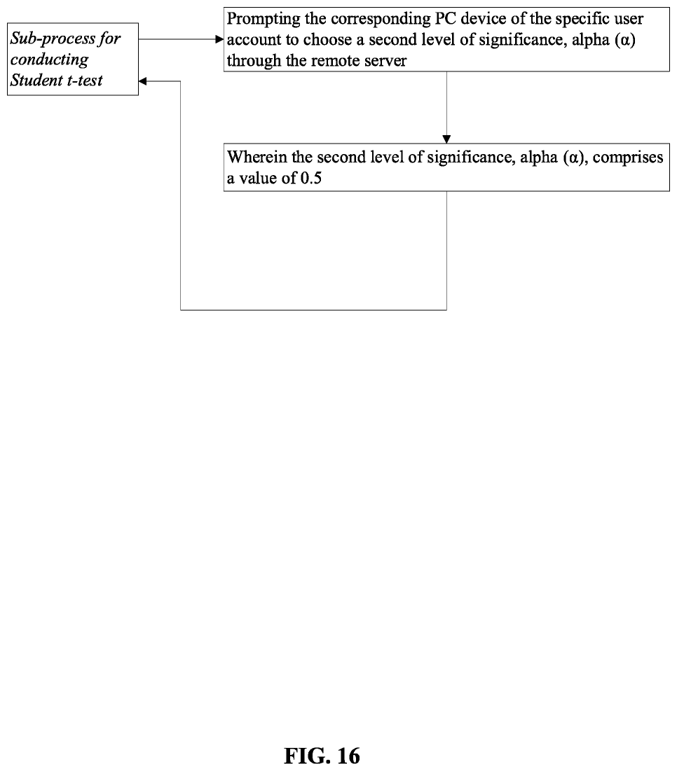

As can be seen in FIG. 14, the method of the present invention provides a sub-process for conducting the Student t-test for the specific treatment. More specifically, the method prompts the corresponding PC device of the specific user account to choose an alternative hypothesis, Hₐ, through the remote server in Step F, wherein the Hₐ is: the risk of occurrence for an adverse outcome of the specific treatment under the allegation of negligence is significantly different from the background risk consequent to random chance, and wherein the adverse outcome was a direct result of the specific treatment which deviates from standards of care. As can be seen in FIG. 15, in an alternative embodiment of the present invention, the sub-process of the method calculates the p-value using the t-value for the one-sample Student t-test through the remote server. The method subsequently accepts the null hypothesis, H₀, if the p-value is greater than or equal to α, wherein the risk of occurrence for an adverse outcome of the specific treatment is not significantly different from the background risk of the hypothetical treatment. Otherwise, the method rejects the null hypothesis, H₀, in favor of the alternative hypothesis Hₐ, if the p-value is less than α, wherein the risk of occurrence for an adverse outcome of the specific treatment is significantly different from the background risk of the hypothetical treatment. As can be seen in FIG. 16, in another embodiment of the present invention, the sub-process of the method prompts the corresponding PC device of the specific user account to choose a second level of significance, alpha (α) through the remote server in Step F through the remote server, wherein the second level of significance, alpha (α), comprises a value of 0.5. More specifically, the method states the alternative hypothesis, Hₐ, is always: “Under the circumstances specific to the allegation of negligence, the risk of the occurrence for an adverse outcome consequent to treatment is significantly different from background risk of its occurrence consequent to random chance. Therefore, the adverse outcome was a direct result of treatment and a departure from standards of care” In terms of the scientific method, Hₐ≠μ.

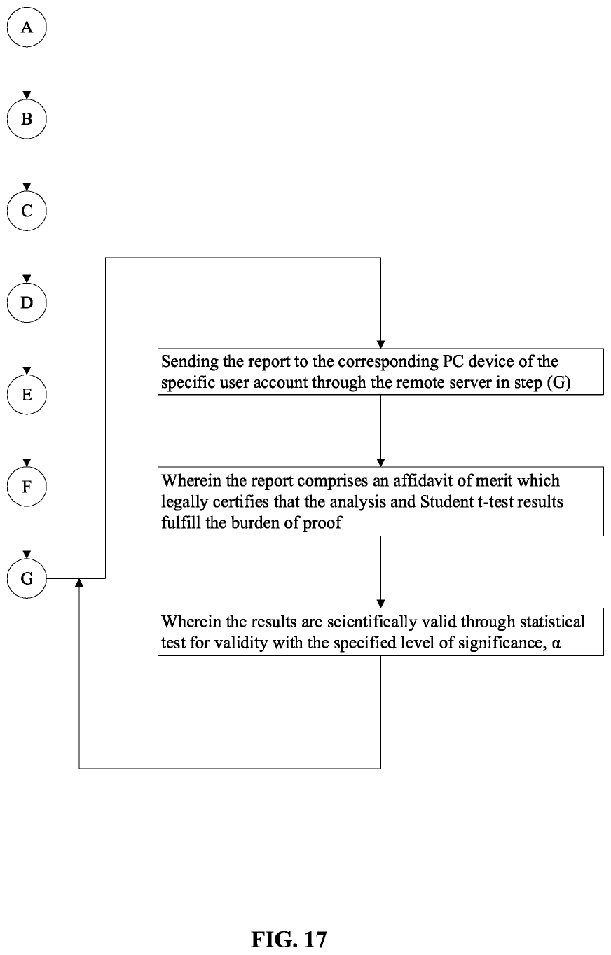

As can be seen in FIG. 17, the method of the present invention provides a sub-process for reporting the Student t-test results for the specific treatment. More specifically, the method sends the report to the corresponding PC device of the specific user account through the remote server in Step G, wherein the report comprises an affidavit of merit which legally certifies that the analysis and Student t-test results fulfill the burden of proof, and wherein the results are scientifically valid through statistical test for validity with the specified level of significance, α.

Although the scientific method was accepted by scientists since Sir Francis Bacon and statistical analysis was the gold standard of science for 100 years, experts never used them to prove or disprove the “null hypothesis.” Until now, jurors, judges and lawyers could not know the difference between an expert who is 95% certain and one who is 50.01% certain. Now, they have a way. The present invention provides an efficient and effective method to exploit the ambiguity inherent in a reasonable degree of medical certainty. In fact, any intentional effort to do so is exposed. Also, the lower or higher the p-value is relative to alpha (0.05) the more certain are the results. Now, certainty can be optimized and when jurors understand this, they can better fulfill their obligation about the preponderance of the evidence.

The present invention comprises a predictive model, which has three parts: (1) The method: The scientific method and algorithm employs a statistical test, which fulfills and exceeds a preponderance of the evidence, to objectively prove or disprove, the “null hypothesis” that the occurrence of an alleged injury equals random chance. (2) A cost driver: It is a diagnostic tool measuring the damage from a single error-prone opinion as “dollars (avoidable costs) per error-prone opinion.” (3) Existing statutes in the law: These are: the Daubert decision that permits scientific opinions proven by the scientific method to be admissible as evidence; Rule 11 that excludes frivolous legal pleadings and penalizes those who make them, and affidavits of merit or expert witness reports which certify that expert opinions meet the burden of proof.

A. The Method

Although all specialty organizations have ethical guidelines for objective medical testimony, they do not focus on the principles or methodology of this forensic decision-making to demonstrate how and why an expert objectively arrived at an opinion. Thus, the first step in this predictive model uses the scientific method to define the heuristics for the statistical validation of an opinion. The purpose of the scientific method is to examine the influence of error on conclusions. Specific to medical malpractice, it does so, not by testing the hypothesis that alleges negligence, called the alternative hypothesis, but, rather, by testing the hypothesis, which is always the proposition that there is no negligence, called the null hypothesis.

B. The Cost Driver

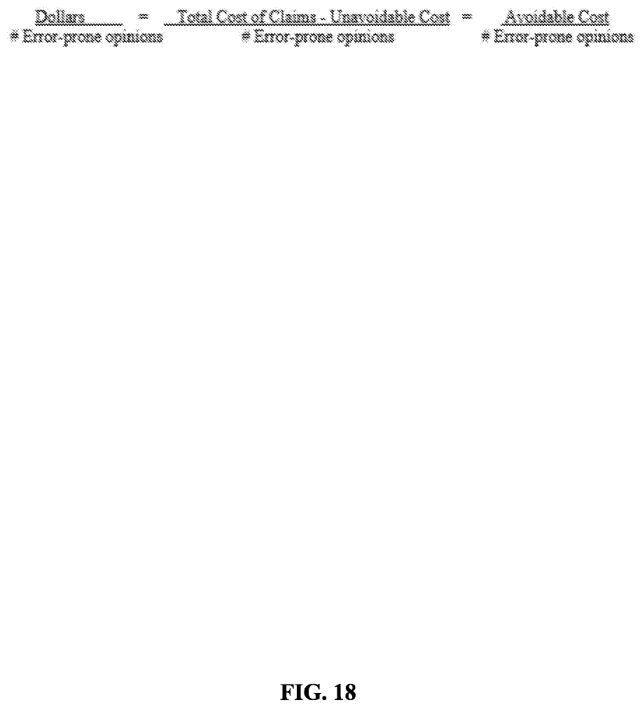

In general, a cost driver is a very powerful analytical tool that serves to make business decisions that are necessary for the survival of a company. It considers all activity costs arising from production divided by some attribute that is common to all those activities. It is a diagnostic signal; the higher the cost driver the higher the price. In the case of medical professional liability insurance, the cost driver is also a diagnostic signal for all the activity costs that contribute to uncertainty. The higher the cost driver the greater the uncertainty. Uncertainty is the unpredictability of avoidable costs. The single attribute common to avoidable costs is the error-prone opinion. Therefore, the cost driver is “dollars per error-prone opinion.” (FIG. 18) It focuses attention on avoidable costs arising from miscarriages in the tort system, such as: excessive legal processes, misinformed verdicts and unjustified awards, caused by a single error-prone opinion on either side of a case.

Although cost drivers were computed ever since there was cost accounting, using it for this purpose was never considered. The value of the cost driver was not to be underestimated. Until now, medical professional liability carriers used predictive models, such as the Predictive Risk Of New Event (PRONE) Score. Common to these models were metrics linking certain attributes of individual providers or institutions, such as: age, gender, specialty, location, frequency of complaints, patient satisfaction, etc., to risks of recurrent claims. Such models used complicated heuristics showing how these attributes contributed to departures from standards of care. However, departures from standards of care were not the root cause of the crisis. Predictive models that held certain attributes of providers and hospitals responsible for the crisis, can only lead to business decisions about management of those attributes. None of these attributes directly impacted cost of claims. Now, the cost driver, was a diagnostic signal for how one attribute, error in an expert's opinion, influenced the cost of claims. Error was the root cause of the crisis. The cost driver, that held error responsible, would lead to business decisions about the management of those attributes that exploited error in an opinion. As a result, management of error-prone opinions on both sides of a malpractice case could virtually eliminate avoidable costs. Whatever was measured was managed.

C. Existing Statutes in the Law

There are three statutes in existing law that make this predictive model workable: the Daubert Decision, Rule 11 and the requirement for expert reports and affidavits of merit.

(1) The Daubert Decision: In Daubert vs. Merrell Dow Pharmaceuticals, the United States Supreme Court ruled that, faced with a proffer of expert scientific testimony, the trial judge must make a preliminary inquiry of whether the testimony's underlying heuristic is scientifically valid, has been or can be tested, is consistent with peer review and accepted science, and has a quantifiable error. The focus of such an inquiry is solely on principles and methodology of decision-making, not on the conclusions that they generate. In effect, the Supreme Court of the United States established a precedent that holds expert opinions to the same standards used in the scientific method which includes the level of certainty of 95%, a value that is the sine qua non of peer review and accepted science. A presiding judge would be inclined to consider expert opinions that rely on the algorithm as admissible evidence and disinclined toward a less objective approach.

The problem with the Daubert decision was it allowed certain evidence that may be “good science” but was not generally accepted. To avoid this, some states used the Frye test, or general acceptance rule, for admissibility because it relied on general standards commonly accepted by the scientific community or other expert witnesses. However, if “bad science” was generally accepted, the Frye test may allow it. In actuality, the much-heralded shortcoming in the Daubert decision was much ado about nothing. Nevertheless, in medical malpractice it was generally accepted that standards of care were generally accepted.

With this algorithm operational, the focus is on principles and methodology of decision-making, not results. This is fundamental to the Daubert decision. The heuristic, itself, is generally accepted scientific principles. The irony of ironies is, Daubert was decided because generally accepted “bad science” was, in fact, admitted in Daubert vs. Merrell Dow Pharmaceuticals. If this heuristic was performed, it would have retained the null hypothesis; the case would never get to the Supreme Court and there would be no Daubert decision.

Although decided in 1993, Daubert was used inconsistently. Until now, a presiding judge could be challenged when using the Daubert decision. Now, judges are afforded the advantage to use it in the way it is intended, i.e., to admit evidence that is scientifically valid with 95% confidence, that has been or can be tested with 95% confidence, that is consistent with peer review and accepted science, because the heuristic is the scientific method, and that has a quantifiable error, only a 5% risk of a type-1 error.

(2) Rule 11: Rule 11 of the Federal Rules of Civil Procedure establishes conditions so that, when filed, lawsuits are valid. Pleadings to the court certify that a lawsuit is filed only after a reasonable investigation of the facts determines that the allegations have evidentiary support or are likely to have evidentiary support after discovery. Rule 11 also specifies the conditions for filing a response to a complaint. The filing of a response certifies to the court that, after a reasonable investigation of the facts, the denials of factual contentions are warranted on the evidence.

The problem with Rule 11 was that, until now, even invalid filings can evade it. Error in an opinion, alone, is not enough to constitute a frivolous claim of negligence. According to Rule 11, a frivolous lawsuit is one that is invalid, yet, is filed or is litigated for improper purposes even when an attorney has knowledge that there is no evidentiary support. Therefore, there must be intent. Frivolous lawsuits have sanctions. However, lawyers on both sides are not obliged to be objective, are zealous advocates for their clients and are able to finesse the threshold of a reasonable degree of medical certainty to the advantage of their clients. Consequently, because there is no way of objectively showing that an attorney is crossing the line by intentionally ignoring error, it is difficult to determine intent. Unless attorneys ignore error with overt impunity, Rule 11 can be confounded.

The same could be said about medical experts. Although obliged to be objective and sworn to be truthful, the authority and credentials of medical experts create a façade of credibility for their testimonies, which can obscure flaws and errors whether introduced intentionally or not. Therefore, unless they introduce flaws and errors with impunity, their testimonies are regarded as a legitimate intellectual difference of opinion and they, too, are not held accountable.

With this algorithm operational, Rule 11 can be used as intended because crossing the line between objectivity and the intentional distortion of the facts is exposed. All flaws and sources of error become patently obvious in the algorithm by a propensity to exaggerate or underestimate risks and from rationalizations to justify these distortions.

Although there have been Federal Rules for Civil Procedures since 1938, these outcomes were never possible. Until now, 50.01% certainty was good enough to cause some frivolous claims to escape attention. Now all frivolous claims and frivolous defenses of negligence can be shown with 95% certainty to be nothing more than mere insinuations of a departure from standards of care.

(3) Expert Reports and Affidavits of Merit: The final step of the algorithm, itself, is the preparation of an expert report or an affidavit of merit. This is also the first step in malpractice litigation. There are ample precedents for such documents. Some states, such as Pennsylvania, require reports from experts on both sides of a case. Other states require affidavits of merit from medical experts before a lawsuit could even be filed.

The problem with expert reports and affidavits of merit was that, until now, they only certified that the opinions of medical experts were proffered to a reasonable degree of medical certainty. Opinions met the burden of proof but fell short of any real scientific validation. The differences between the two expert reports or affidavit of merits was considered just as honest intellectual differences between medical experts.

With this algorithm operational, all finders of facts involved in discovery have the opportunity to objectively examine the validity of an expert's opinion expressed in the report or affidavit of merit based on how the expert adhered to the heuristics of the algorithm. From the very start of a case, decisions of whether or not to support those opinions can be made, not as a matter of legal tactics but, rather, as standards of the law.

A presiding judge would not wantonly admit evidence in a certified affidavit of merit or expert report that can be shown to be intentionally error-prone in its content by a propensity to exaggerate variables in order to prove or disprove the null hypothesis. The intentional exaggeration of variables goes beyond type-1 error even when an expert uses α=0.5, rather than 0.05 in the heuristic. Even when properly executed, whatever the level of significance is. the judge may admit both as evidence leaving it to the jury to decide. However, the judge has an obligation to instruct the jury about the difference between the two opinions.

A plaintiff attorney cannot wantonly litigate a lawsuit knowing that it is founded on objectively proven invalid evidence in an expert's report. To do so, fulfills the criteria for a frivolous lawsuit and invites sanction. It also sets conditions for defendants to sue plaintiff attorneys for malicious prosecution after defendants prevail in invalid malpractice cases.

A defense attorney would not wantonly risk exposing a client to an excessive verdict consequent to an error laden opinion in a report certified by the expert. Under these circumstances, recommending an early settlement is the most appropriate course of action.

An expert, too, would not wantonly abuse his or her obligation. The language certifies to the court that the opinion is not only expressed to a reasonable degree of medical certainty but, also, is scientifically valid, has been and can be tested for validity, is consistent with peer review and accepted science and has been objectively examined for error. Now, it is not just intellectual differences between experts but the differences in content. It is, also, the difference between 5% type-1 error and 50% type-1 error. Although expert reports and affidavits of merit were around for many years, they were underestimated. Until now, they were just catalysts that started the reaction but were not part of it. Now, these documents unify the predictive model and are, very much, a part of it. Unlike before, an expert's signature on these documents is not a mere formality.

### Acceptance of the Strategy

There is a counterpart to the cost driver. It is the diagnostic signal for what medical professional liability carriers do right. For the lack of a better term, call it “the benefit driver.” Unavoidable cost÷the number of valid opinions=dollars per valid opinion. As will be shown later, each valid opinion is always far less costly than an error-prone opinion. This has obvious implication, especially for medical professional liability companies. They contract with defense lawyers who, in turn, retain defense medical experts. Because a medical professional liability company has an obligation to control cost of claims, it is in its interest to include a provision in the standard contract with a defense attorney that requires any medical expert retained by that attorney to use heuristics of forensic decision-making consistent with the scientific method. During an evidentiary hearing, a defense expert's use of this heuristic raises the question for the presiding judge of why the heuristic was not used by the plaintiff expert? The lawyer, to whom that question is directed, is at a distinct disadvantage. Rather than having credibility challenged, it will behoove plaintiff attorneys to also require their experts to use the heuristic.

Since 2018, a disturbing trend of record-breaking jury verdicts, gives this heuristic greater relevance. There are a number of factors explaining this trend but the most pernicious are a sympathetic jury pool and an emerging class of aggressive plaintiff attorneys. Regardless of how sympathetic the jury pool is, they still swear to be objective. Even the most aggressive plaintiff attorney still must obey the Federal Rules of Civil

Procedure. The use of this heuristic by expert witnesses on both sides can and will modify the behavior of an overly aggressive attorney and an overly sympathetic juror.

This predictive model does not replace traditional adversarial proceedings; it improves them. First, experts arrive at opinions, except they prove their hypothesis using the scientific method. Next, experts prepare affidavits of merit or expert witness reports certifying their proof as evidence, except they send a clear signal about who is 95% certain or who is 50.01% certain and which reports are a product of an expert's judgement about the preponderance of the evidence and which were statistically tested for error using the scientific method.

Then, judges, in evidentiary hearings, review these filings and decide which evidence is, or is not, admissible, except they know that the underlying heuristic of the filing employed the scientific method. They also decide which pleadings are, or are not, frivolous and, if so, who should be penalized, except they know that the said heuristic also exposes any attempt to exploit error.

Next, comes discovery. Depositions, status conferences, settlement hearings and pretrial conferences proceed as usual except, because of the heuristic, they do what they are intended to do—weed out error. Finally, if no remedy is found during discovery, a jury will hear exactly the same evidence, now thoroughly scrutinized for error, except, when knowing which expert witness was 95% certain and which was 50.01%, jurors are unlikely to be influenced by the ambiguity in “the preponderance of the evidence.

### Whatever Gets Measured Gets Managed—Eliminating Avoidable Costs

When operational, not only is justice better served but, as importantly, the cost driver becomes a powerful analytical tool for every medical professional liability company in the country.

As the example: NCRIC, Inc., an AM Best “A” rated medical professional liability insurance company in Washington D.C., was chosen for the following analysis for several reasons. AM Best “A” rating being the 3^{rd} highest rank given to secure/excellent companies. NCRIC was a small reciprocal company with total earned premiums of $19.6 million. It insured 4700 physicians in Virginia, Maryland, Delaware, North Carolina and West Virginia, but was the major carrier serving doctors in the District of Columbia, where earned premiums were $6.7 million. The analysis is confined only to NCRIC's 2001 litigation experience in the District of Columbia. Its claims experience elsewhere, and other extenuating circumstances are not considerations. The District of Columbia represented the perfect storm.

(1) At that time, NCRIC insured 62% of doctors in the District of Columbia; Hartford was the only other carrier. Therefore, there was limited availability to other coverage.

(2) High-end insurance premiums were $140,000 a year, making medical professional liability insurance unaffordable.

(3) Because of issues of availability and affordability, some doctors chose early retirement, some abandoned competencies like obstetrics and some relocated to Maryland and Virginia, making some medical services less accessible.

(4) There was no tort reform in the District of Columbia, no cap on noneconomic damages and lawyers accepted cases on contingency.

(5) The severity of claims in Washington, D.C. was the highest in the nation.

Unfortunately, NCRIC succumbed to this crisis and stopped operating in 2005. It was one of a very few AM Best “A” rated medical professional liability insurance companies in the country to fail.

The following sensitivity analysis of NCRIC's financial statement demonstrates the use of the predictive model. Data are obtained from the litigation experience before NCRIC closed. The litigation experience is most complete for 2001. Even so, some data pertaining to the District of Columbia in NCRIC's financial report are commingled with losses from other states. To compensate, this analysis extrapolates 2001 data from the National Practitioner Data Bank, the Council of Economic Advisers and the American Academy of Actuaries.

NCRIC's 2001 data for the District of Columbia showed that it tried 20 cases and settled 2 others. The 10:1 ratio of litigated to settled cases in NCRIC's data was noteworthy when compared to the opposite industry-wide trend and suggested an internal disposition for NCRIC not to settle cases. Of the 20 cases litigated, 5 (25%) resulted in plaintiff verdicts, 13 (65%) ended in defense verdicts and 2 ended in a mistrial or a hung jury.

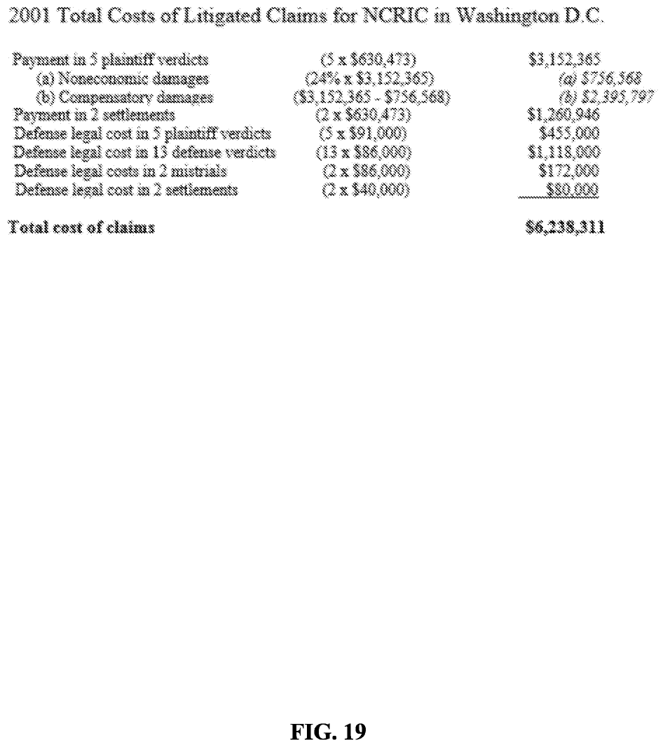

The total costs of awards and settlements in the District of Columbia were not clear in NCRIC's financial report. However, in data from the National Practitioner Data Bank, $630,473, was the mean award for 5 plaintiff verdicts and 2 settlements in the District of Columbia, which was the highest in the nation. (FIG. 19) Also, important to note was that only one of the five plaintiff verdicts exceeded NCRIC's reinsurance threshold of $500,000, four no greater than $500,000 and one no less than $1.15 million. This will have relevance later.

The costs of compensatory versus noneconomic damages were also not clearly stated in NCRIC's financial report. However, data from the Council of Economic Advisers showed that, in 2001, non-economic (punitive) damages awarded for malpractice cases in the District of Columbia were 24% of plaintiff verdicts. (FIG. 19)

Likewise, the total legal transaction expenses were not clearly stated in NCRIC's financial report. However, data from the American Academy of Actuaries showed that, in 2001, for the District of Columbia, total defense costs averaged $86,000 per claim when the defendant prevailed at trial, $91,000 when the plaintiff prevailed, $40,000 when a suit was settled and $17,000 when a case was dismissed.

From these combined sources, it was estimated that the total cost of claims for NCRIC in Washington D.C. was $6,238,311. (FIG. 19) Earned premiums in Washington D.C. were $6.7 million, Therefore, in the District of Columbia, NCRIC's the claims to premium ratio was 0.925.

In the 22 cases decided in 2001, there are 44 opinions, one on each side of a case. Since these were never examined, there is no way of determining how many were error-prone. Although Brennan, et al, implies a high likelihood of potential error-prone opinions among all cases filed, this analysis makes the assumption that 90 percent of the opinions behind the NCRIC experience made valid assertion and, if tested by the above methodology, were not distorted by extremes. Therefore, 10 percent, or only four opinions, patently exploited error for either side. Of these four error-prone opinions, it is further assumed that two are from plaintiff experts and two are from defense experts, which gives equal weight to misleading assertions on both sides of a case. Lastly, it is assumed that the two plaintiff opinions and the two defense opinions would result in misinformed jury verdicts if they were adjudicated. These four error-prone opinions are the denominator of the cost driver equation.

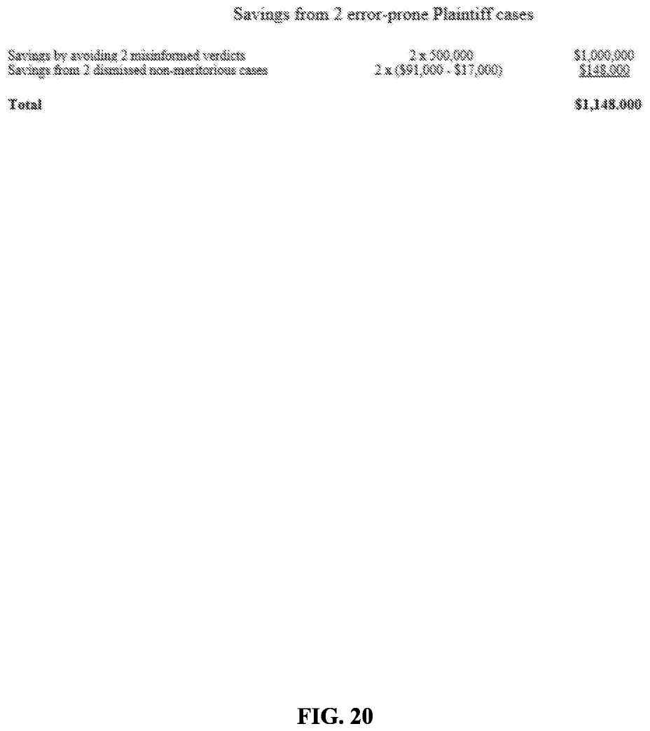

If the two objectively proven error-prone opinions of plaintiff experts were impeached during discovery, these cases would result in dismissals. Under these conditions, the two plaintiff attorneys and the two plaintiff experts would not risk the consequences of intentionally continuing what could now be objectively proven as frivolous lawsuits under Rule 11. This saved NCRIC $1 million in potential verdicts (assuming that the single $1.15 million verdict was a meritorious case) and avoids the transaction cost of going to trial, $91,000 per case. However, the transaction cost for dismissal was $17,000 per case, an unavoidable cost. If this was done, NCRIC would have saved $1,148,000. (FIG. 20)

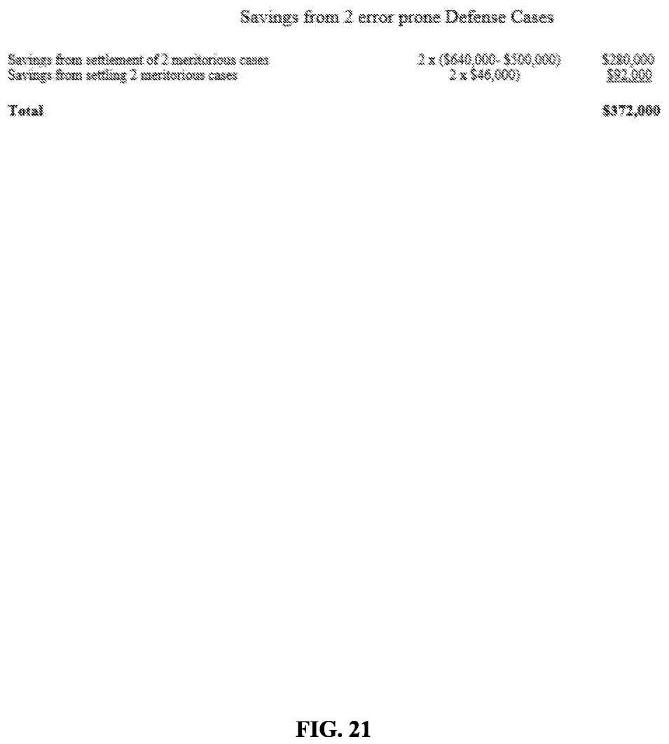

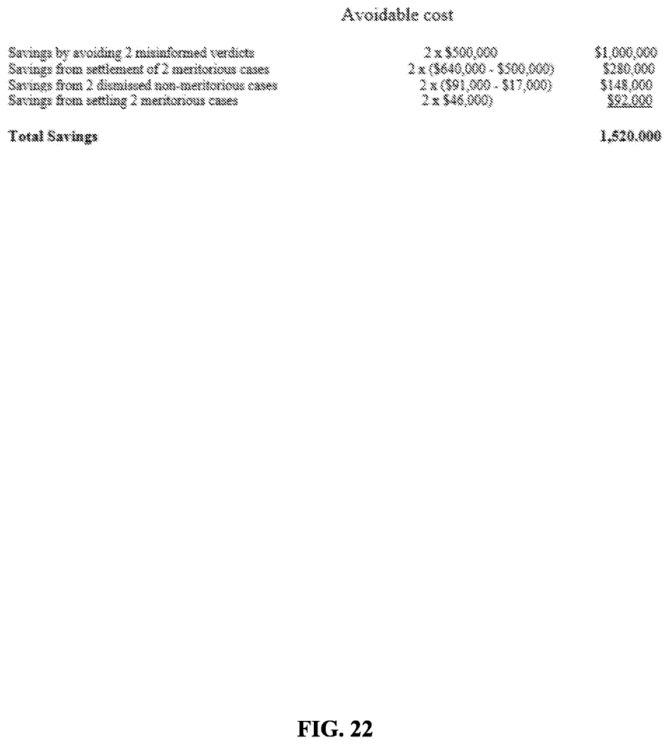

If the two objectively proven error-prone opinions of defense experts were impeached during discovery, it would be in NCRIC's best interest to seek the earliest possible settlement of these cases because a jury might be inclined to award an excessive judgement. Assuming that the settlement values were in the neighborhood of $800,000, expedient settlements had two advantages. First, it decreased transaction costs for the defense from $86,000(cost for trial) to $40,000(cost for settlement), an avoidable cost of $46,000 per case. Second, because it took more than a year for a case to appear on a docket, plaintiffs may be amenable to accept settlements of 80% of their potential values, or $640,000. Because, likely, these were among those 4 cases in the NCRIC data that had a verdict of no greater than $500,000, the savings for each case was $140,000. The presumed $500,000 verdicts were unavoidable costs. Nevertheless, NCRIC, still, would have saved $372,000 of avoidable costs. (FIG. 21). As a consequence of exposing error on both sides, the saving to NCRIC could be $1,520,000. (FIG. 22).

Avoidable costs were $1,520,000, the numerator in the cost driver equation. Unavoidable costs were: $6,238,311−$1,520,000=$4,718,311. They were inescapable, arising from proper function in the tort system. No legitimate award was overturned by this analysis and justice was served. Now, all the factors in the cost driver equation were identified. Therefore, the cost driver for uncertainty was $1,520,000÷4 error-prone opinions=$380,000 per error-prone opinion. In 2001, a single error-prone expert's opinion cost NCRIC $380,000. By eliminating these 4 error-prone opinions during early procedures, NCRIC could have saved $1,520,000. That is 24 percent of total costs of claims. The claims to premium ratio would be 0.7.

As an aside, the aforementioned “benefit driver” is $4,718,311(unavoidable cost)÷40 valid opinions =$117,958 per valid opinion. Each error-prone opinion is $262,042 more costly than a valid opinion.

Admittedly, this is a retrospective sensitivity analysis and a true prospective study using the predictive model will produce different results, but, without question, a great deal of money would still be saved. Also, it was entirely possible that more than 4 error-prone opinions could be found if this was a prospective study.

In the final analysis, a 24 percent savings is, by any account, remarkable. Even if there was tort reform in the District of Columbia capping non-economic (punitive) damages at $250,000 per claim, tort reform, alone, would not accomplish this. First, such tort reform only effects the cost of punitive damages in a judgment and has nothing to do with transaction costs. Second, it was unclear how these punitive damages were awarded among the 5 plaintiff verdicts. As noted earlier, only one judgement exceeded the reinsurance threshold of $500,000, therefore, the 5 plaintiff verdicts must include 4 no greater than $500,000 and 1 no less than $1.15 million. Total punitive damages were $756,568. Punitive damages are common in allegations of medical negligence in the District of Columbia and, if awarded in each case, the $756,568 would be proportionately divided among these 5 plaintiff verdicts as 24% of each, this according to the aforementioned data from the Council of Economic Advisers. Under these circumstances, a $250,000 cap per claim only saved $26,000 or 0.4 percent. Even if the entire punitive damages were awarded against a single case, savings would be $506,568 ($756,568−$250,000), or 8 percent.

Actuaries could hardly be faulted for being risk-adverse with these calculations. On Aug. 2, 2005, NCRIC was acquired by ProAssurance and closed down. NCRIC stock was valued at 25 cents on the dollar. The claims to premium ratio was 1.028. Today, in the District of Columbia, the premium for high-risk specialties is over $150,000. None of this is good news. Diagnostic signals that the cost driver sends about the exploitation of error on both sides of malpractice cases are impossible to ignore.

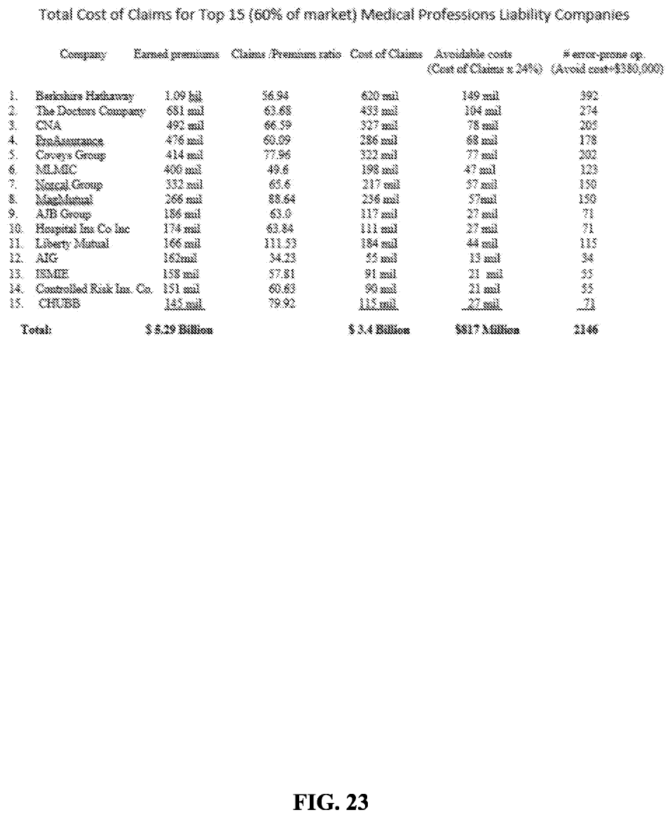

Knowing this now, makes it inconceivable that the cost driver would not be a standard subject on the agendas of board of director meetings in every medical professional liability company in the country. There is current data on the nation's top fifteen medical professional liability insurance carriers. (FIG. 23) The lowest claims to premium ratio is 34.23%; the highest ratio is 111.53%. The mean is 66.7%; the median is 63% and the mode is 63.4%. These data represent the industry standard before the use of the predictive model. Absent more data, cost drivers cannot be determined. However, assuming the cost driver and savings are the same as the NCRIC data, there are a total of 2146 error-prone opinion and a total saving of $817 million, which are the total avoidable costs. The mean avoidable cost is $54.5 million, the median is $44 million and the mode is $51 million. The mean claims to premium ratio ise now 50.2%—perhaps a new industry standard.

The industry standard became the standard before this predictive model was used. The estimated outcome in these 15 top carriers resulted from a cost driver of $380,000/error-prone opinion and a savings of 24%. They may be greater. Nevertheless, these estimates happen because of differences in opinions between experts. Those who render opinions with a 50% risk of type-1 error result in a mean claims to premium ratio of 66.7% and those who render opinions with a 5% risk of type-1 error result in a mean ratio of 50.2%. Whatever gets measured gets managed.

Conclusion:

Is this too good to be true? There is no better way to answer this question than to test the null hypothesis of the predictive model, itself. The null hypothesis is: “this predictive model does not change the average cost of a claim.” The mean, μ, is the average cost of a claim. For the purpose of this test, μ, is $6,238,311÷22=$283,560. The formula for the null hypothesis is Hₒ=μ. The alternative hypothesis is: “this predictive model decreases the average cost of a claim.” The formula for the alternative hypothesis is Hₐ<μ.

The independent variables for both the null hypothesis and alternative hypothesis include: the scientific method, the Daubert decision, Rule 11, affidavits of merit, the cost driver, expert witnesses, opinions and adversarial procedures. Because the null hypothesis is unchanged, all the independent variables in it have the relative value of 1.0. Next is assumed that when the predictive model is used on each independent variable in the Alternative hypothesis, the cost of a claim is decreases by an extremely modest 1.0 percent, having a relative value of 0.99. The rationale supporting this assumption is that any opinion can be impeached just by subjecting it to random arguments. However, the power of the scientific method and statistical analysis should be able to do so at least 1% better.

The observed cost of a claim for any independent variable in the alternative hypothesis is 0.99×$283,560=$280,725. Using the one sample t-test, the eight values for the observed cost of a claim, $280,725, are placed in the “sample” data field; μ is $283,560 and α is 0.05. The resulting p-value is 0.00001. The null hypothesis is clearly and convincingly rejected.

Regardless, of how convincing this may be, paradigms never change without conflict because they threaten the status quo. However, the status quo is the crisis. Some might object because this predictive model threatens adversarial procedures; however, as shown, it does not. Some might object because it threatens their ideology. They see this as tort reform, which is incompatible with their ideology. Technically, this is not; this is policy. The final, and most desperate, objection of some will be the “social justice” objection. This predictive model is a shameful conspiracy by greedy, selfish and dishonest forces in the private sector, namely physicians and malpractice carriers, to avoid the consequences of malpractice on poor, unsuspecting victims. Whatever their objections might be, no longer matter because leadership in the private sector will understand this predictive model as unstoppable. That is why it is called a paradigm—an idea that looms above all others.

The very first time a presiding judge holds both sides accountable to the same algorithm, will be the precise moment the algorithm becomes the standard and the paradigm begins. In that paradigm will be reporting systems for medical errors and special medical malpractice courts. A data base will categorize and standardize the percent increase of risk from differences in treatment observed between corresponding phases in the algorithm. Expert witness will use this data to render opinions and will be trained and certified before offering opinions. A more certain burden of proof than a preponderance of the evidence, specifically, clear and convincing evidence, will be accepted so that jurors can make decisions with greater certainty than the flip of a coin. The crisis will be over. The paradigm makes medicine great again. It, also, makes law great again.

Some propose another paradigm—socialized medicine. Socialized medicine makes every physician a federal employee. Whether it makes anything great again is another topic; nevertheless, there will be no need for medical professional liability insurance. If things happen, and they will, the government will provide protection. It could be argued this effectively ends the malpractice crisis, except for one thing—the Federal Torts Claims Act. This law gives a private party the right to sue a federal employee for medical negligence. Under these circumstances, all costs and threats fall squarely on the government. Socialized medicine does not end the crisis, it just redistributes the disruption.

Although, no one knows what the future brings, it is absurd to continue what this crisis already brought for 40 years. This “new normal” was permitted with complete disdain of consequences, not the least of which is the cost. The $60 billion includes $45 billion in defensive medicine. The costs of defensive medicine are incurred from the mere insinuation of liability and are essentially avoidable costs passed on to everyone. Other avoidable costs are the nationwide avoidable costs for all medical professional liability insurance companies. Among these are the aforementioned top 15, representing 60% of the market, plus an additional 40 other carriers. If, as shown in FIG. 23, $817 million are 60% of these avoidable costs, then, 100% is $1.4 billion. The corrected national cost of the crisis $46.4 billion: $45 billion (defensive medicine)+$1.4 billion (nationwide carrier avoidable cost), all avoidable. This corresponds to $155 from every man, woman and child in the country per year—$1.86 trillion over the last 40 years—the personal impact caused by all this disruption.

If not bad enough, what is the cost driver? Until now, in courtrooms all over the country, all error-prone opinions that sway the minutest quanta of certainty above 50% has the impact of $46.4 billion a year to our nation. The root cause of the crisis is error-prone opinions by medical experts. Nationwide there are 3684 error-prone opinions: $1.4 billion (nationwide avoidable costs for all liability carriers) +$380,000 (avoidable cost per error-prone opinion from NCRIC data). Therefore, the national cost driver is $12.6 million per error prone opinion: $46.4 billion (corrected cost of crisis) +3684 error-prone opinions. This is a diagnostic signal to the nation. Knowing this makes it beyond absurd to do nothing. Today is the tipping point.

Although the scientific method, the cost driver and existing statutes in the law were “shovel ready” for 40 years, their values were unrealized. The focus remained on the disruption the crisis caused rather than on the cause. The “the new normal” or the Cloward-Piven strategy, whatever the case may be, badly destabilized the healthcare system and produced a tort system that was unable to discriminate between an opinion with a 50.01% certainty and an opinion with a 95% certainty.

That was then; this is now. Today, the scientific method, the cost driver and existing statutes in the law are the three components of the predictive model and their values are irrefutable, self-evident and inescapable. Stakeholders and institutions in the private sector: doctors, lawyers, judges, expert witnesses, liability companies, hospitals, patients, the list goes on, will come to regard this predictive model as the game changer. What happens next is not left to politicians. Implementation depends more on these stakeholders and institutions than it does on government. This, indeed, is a paradigm shift. It is time to make medicine great again.

Although the invention has been explained in relation to its preferred embodiment, it is to be understood that many other possible modifications and variations can be made without departing from the spirit and scope of the invention as hereinafter claimed.

What is claimed is:

1. A method and system for providing statistical analysis of a medical treatment comprising the steps of:
(A) providing a plurality of user accounts managed by at least one remote server, wherein each of the plurality of user accounts is associated with a corresponding personal computing (PC) device;
(B) prompting the corresponding PC device of a specific user account to enter a medical treatment under allegation of negligence through the remote server;
(C) creating a hypothetical treatment for the specific medical treatment through the remote server, wherein the hypothetical treatment is created following the standards of care;
(D) determining the background risk for the hypothetical treatment through the remote server, wherein the background risk comprises the inherent random occurrence of the hypothetical treatment;
(E) conducting an analysis of the specific medical treatment to determine a relative risk for each of a plurality of phases comparing with the hypothetical treatment, wherein the specific treatment comprises the plurality of phases, and wherein the resulting relative risks are grouped into a sample of risk data;
(F) conducting a one-sample Student t-test for risk data of the specific treatment, wherein the null hypothesis, H₀ is: the risk of occurrence for an adverse outcome of the specific treatment under the allegation of negligence is not significantly different from the background risk consequent to random chance, and wherein the level of significance, alpha (α), is 0.05; and
(G) reporting the analysis of the specific medical treatment under allegation of negligence to the corresponding PC device of the specific user account through the remote server, wherein the report comprises the statistical Student t-test results and conclusion.

2. The method and system for providing statistical analysis of a medical treatment as claimed in claim 1 comprising the steps of:
separating the specific treatment into a plurality of phases in step (E) through the remote server;
investigating each phase of the specific treatment for any deviation from the corresponding phase of the hypothetical treatment;
determining a risk factor based on the comparison for each phase of the specific treatment; and
Calculating the relative risk for each phase, wherein the relative risk is the product of the risk factor and the background risk of the hypothetical treatment.

3. The method and system for providing statistical analysis of a medical treatment as claimed in claim 2 comprising the steps of:
investigating a presentation phase of the plurality of phases for both the specific and hypothetical treatments through the remote server; and
wherein the presentation phase comprises the circumstance and condition relevant to the initial encounter of the specific medical treatment.

4. The method and system for providing statistical analysis of a medical treatment as claimed in claim 2 comprising the steps of:
investigating a pre-treatment phase of the plurality of phases for both the specific and hypothetical treatments through the remote server; and
wherein the pre-treatment phase comprises details of the medical work-up of the specific medical treatment.

5. The method and system for providing statistical analysis of a medical treatment as claimed in claim 2 comprising the steps of:
investigating an evaluation phase of the plurality of phases for both the specific and hypothetical treatments through the remote server; and
wherein the evaluation phase comprises the interpretation of lab results, imaging studies, etc.

6. The method and system for providing statistical analysis of a medical treatment as claimed in claim 2 comprising the steps of:
investigating a diagnostic phase of the plurality of phases for both the specific and hypothetical treatments through the remote server; and
wherein the diagnostic phase comprises the diagnosis of a medical treatment.

7. The method and system for providing statistical analysis of a medical treatment as claimed in claim 2 comprising the steps of:
investigating a discrimination phase of the plurality of phases for both the specific and hypothetical treatments through the remote server; and
wherein the discrimination phase comprises the most appropriate treatment among therapeutic alternatives available.

8. The method and system for providing statistical analysis of a medical treatment as claimed in claim 2 comprising the steps of:
investigating an informed consent phase of the plurality of phases for both the specific and hypothetical treatments through the remote server; and
wherein the informed consent phase comprises the education and communication to a patient to make informed decision about available treatment options.

9. The method and system for providing statistical analysis of a medical treatment as claimed in claim 2 comprising the steps of:
investigating a selection phase of the plurality of phases for both the specific and hypothetical treatments through the remote server; and
wherein the selection phase comprises the choice of treatment from the available treatment options.

10. The method and system for providing statistical analysis of a medical treatment as claimed in claim 2 comprising the steps of:
investigating a technical phase of the plurality of phases for both the specific and hypothetical treatments through the remote server;
wherein the technical phase comprises the application of a constellation of technical decisions; and
wherein the technical phase comprises the performance of technical details of the chosen treatment.

11. The method and system for providing statistical analysis of a medical treatment as claimed in claim 2 comprising the steps of:
investigating a resolution phase of the plurality of phases for both the specific and hypothetical treatments through the remote server; and
wherein the resolution phase comprises post-treatment follow-up.

12. The method and system for providing statistical analysis of a medical treatment as claimed in claim 2 comprising the steps of:
investigating a discharge phase of the plurality of phases for both the specific and hypothetical treatments through the remote server; and
wherein the discharge phase comprises the circumstance and condition relevant to the final encounter.

13. The method and system for providing statistical analysis of a medical treatment as claimed in claim 1 comprising the steps of:
prompting the corresponding PC device of the specific user account to choose an alternative hypothesis, Hₐ, through the remote server in step (F);
wherein the Hₐ is: the risk of occurrence for an adverse outcome of the specific treatment under the allegation of negligence is significantly different from the background risk consequent to random chance; and
wherein the adverse outcome was a direct result of the specific treatment which deviates from standards of care.

14. The method and system for providing statistical analysis of a medical treatment as claimed in claim 13 comprising the steps of:
calculating the p-value using the t-value for the one-sample Student t-test through the remote server;
accepting the null hypothesis, H₀, if the p-value is greater than or equal to α, wherein the risk of occurrence for an adverse outcome of the specific treatment is not significantly different from the background risk of the hypothetical treatment; and
rejecting the null hypothesis, H₀, in favor of the alternative hypothesis Hₐ, if the p-value is less than α, wherein the risk of occurrence for an adverse outcome of the specific treatment is significantly different from the background risk of the hypothetical treatment.

15. The method and system for providing statistical analysis of a medical treatment as claimed in claim 13 comprising the steps of:
prompting the corresponding PC device of the specific user account to choose a second level of significance, alpha (α) through the remote server in step (F) through the remote server; and
wherein the second level of significance, alpha (α), comprises a value of 0.5.

16. The method and system for providing statistical analysis of a medical treatment as claimed in claim 1 comprising the steps of:
sending the report to the corresponding PC device of the specific user account through the remote server in step (G);
wherein the report comprises an affidavit of merit which legally certifies that the analysis and Student t-test results fulfill the burden of proof; and
wherein the results are scientifically valid through statistical test for validity with the specified level of significance, α.
